SEQUENCE LISTING (16 sequences)

SEQ ID NO: 1 - DNA, 771 bp, Respiratory syncytial virus
atggaaacatacgtgaacaagcttcacgaaggctccacatacacagctgctgttcaatacaatgtcctagaaaaagacgatgaccctgcatcacttacaatatgggtgcccatgttccaatcatctatgccagcagatttacttataaaagaactagctaatgtcaacatactagtgaaacaaatatccacacccaagggaccttcactaagagtcatgataaactcaagaagtgcattgctagcacaaatgcccagcaaatttaccatatgtgctaatgtgtccttggatgaaagaagcaaactggcatatgatgtaaccacaccctgtgaaatcaaggcatgtagtctaacatgcctaaaatcaaaaaatatgttaactacagttaaagatctcactatgaagacactcaaccccacacatgatattattgctttatgtgaatttgaaaacatagtaacatcaaaaaaagtcataataccaacatacctaagatccatcagtgtcagaaataaagatctgaacacacttgaaaatataacaaccactgaattcaaaaatgccatcacaaatgcaaaaatcatcccttactcaggattactattagtcatcacagtgactgacaacaaaggagcattcaaatacataaagccgcaaagtcaattcatagtagatcttggagcttacctagaaaaagaaagtatatattatgttaccacaaattggaagcacacagctacacgatttgcaatcaaacccatggaagattaa

SEQ ID NO: 2 - protein, 256 aa, Respiratory syncytial virus
METYVNKLHEGSTYTAAVQYNVLEKDDDPASLTIWVPMFQSSMPADLLIKELANVNILVKQISTPKGPSLRVMINSRSALLAQMPSKFTICANVSLDERSKLAYDVTTPCEIKACSLTCLKSKNMLTTVKDLTMKTLNPTHDIIALCEFENIVTSKKVIIPTYLRSISVRNKDLNTLENITTTEFKNAITNAKIIPYSGLLLVITVTDNKGAFKYIKPQSQFIVDLGAYLEKESIYYVTTNWKHTATRFAIKPMED

SEQ ID NO: 3 - DNA, 759 bp, Influenza virus
atgagccttctgaccgaggtcgaaacgtatgttctctctatcgttccgtcaggccccctcaaagccgagatcgcgcagagacttgaagatgtctttgctggaaagaacacagatcttgaggctctcatggaatggctaaagacaagaccaatcctgtcacctctgactaaagggattttgggatttgtattcacgctcaccgtgcccagtgagcgaggactgcagcgtagacgctttgtccaaaatgccctcaatgggaatggggatccaaataacatggacagagcagttaaactgtatagaaaacttaagagggagataacattccatggggccaaagaaatagcactcagttattctgctggtgcacttgccagttgcatgggcctcatatacaacaggatgggggctgtgaccactgaagtggcctttggcctggtttgtgcaacctgtgaacagattgctgactcccagcacaggtctcataggcaaatggtggcaacaaccaatccactaataagacatgagaacagaatggttctggccagcactacagctaaggctatggagcaaatggctggatcaagtgagcaggcagcagaggccatggaggttgctagtcaggccaggcaaatggtgcaggcaatgagagccattgggactcatcctagctccagtgctggtctaaaagatgatcttcttgaaaatttgcaggcctatcagaaacgaatgggggtgcagatgcaacgattcaagtga

SEQ ID NO: 4 - protein, 252 aa, Influenza virus
MSLLTEVETYVLSIVPSGPLKAEIAQRLEDVFAGKNTDLEALMEWLKTRPILSPLTKGILGFVFTLTVPSERGLQRRRFVQNALNGNGDPNNMDRAVKLYRKLKREITFHGAKEIALSYSAGALASCMGLIYNRMGAVTTEVAFGLVCATCEQIADSQHRSHRQMVATTNPLIRHENRMVLASTTAKAMEQMAGSSEQAAEAMEVASQARQMVQAMRAIGTHPSSSAGLKDDLLENLQAYQKRMGVQMQRFK

SEQ ID NO: 5 - DNA, 294 bp, Influenza virus
atgagccttctgaccgaggtcgaaacgcctatcagaaacgaatgggggtgcagatgcaacgattcaagtgaccctcttgttgttgctgcgagtatcattgggatcttgcacttgatattgtggattcttgatcgtctttttttcaaatgcatctatcgattctttgaacacggtctgaaaagagggccttctacggaaggagtacctgagtctatgagggaagaatatcgaaaggaacagcagagtgctgtggatgctgacgacagtcattttgtcagcatagagctggagtaa

SEQ ID NO: 6 - protein, 97 aa, Influenza virus
MSLLTEVETPIRNEWGCRCNDSSDPLVVAASIIGILHLILWILDRLFFKCIYRFFEHGLKRGPSTEGVPESMREEYRKEQQSAVDADDSHFVSIELE

SEQ ID NO: 7 - DNA, 1725 bp, Respiratory syncytial virus
atggagttgccaatcctcaaagcaaatgcaattaccacaatcctcgctgcagtcacattttgctttgcttctagtcaaaacatcactgaagaattttatcaatcaacatgcagtgcagttagcaaaggctatcttagtgctctaagaactggttggtatactagtgttataactatagaattaagtaatatcaaggaaaataagtgtaatggaacagatgctaaggtaaaattgataaaccaagaattagataaatataaaaatgctgtaacagaattgcagttgctcatgcaaagcacaacagcagcaaacaatcgagccagaagagaactaccaaggtttatgaattatacactcaacaataccaaaaaaaccaatgtaacattaagcaagaaaaggaaaagaagatttcttggttttttgttaggtgttggatctgcaatcgccagtggcattgctgtatctaaggtcctgcacttagaaggagaagtgaacaagatcaaaagtgctctactatccacaaacaaggccgtagtcagcttatcaaatggagttagtgtcttaaccagcaaagtgttagacctcaaaaactatatagataaacaattgttacctattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacatcatgtaaatgctggtaaatcaaccacaaatatcatgataactactataattatagtgattatagtaatattgttatcattaattgctgttggactgctcctatactgtaaggccagaagcacaccagtcacactaagcaaggatcaactgagtggtataaataatattgcatttagtaactga

SEQ ID NO: 8 - protein, 574 aa, Respiratory syncytial virus
MELPILKANAITTILAAVTFCFASSQNITEEFYQSTCSAVSKGYLSALRTGWYTSVITIELSNIKENKCNGTDAKVKLINQELDKYKNAVTELQLLMQSTTAANNRARRELPRFMNYTLNNTKKTNVTLSKKRKRRFLGFLLGVGSAIASGIAVSKVLHLEGEVNKIKSALLSTNKAVVSLSNGVSVLTSKVLDLKNYIDKQLLPIVNKQSCRISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHHVNAGKSTTNIMITTIIIVIIVILLSLIAVGLLLYCKARSTPVTLSKDQLSGINNIAFSN

SEQ ID NO: 9 - DNA, 1725 bp, Respiratory syncytial virus
atggagttgccaatcctcaaagcaaatgcaattaccacaatcctcgctgcagtcacattttgctttgcttctagtcaaaacatcactgaagaattttatcaatcaacatgcagtgcagttagcaaaggctatcttagtgctctaagaactggttggtatactagtgttataactatagaattaagtaatatcaaggaaaataagtgtaatggaacagatgctaaggtaaaattgatgaaacaagaattagataaatataaaaatgctgtaacagaattgcagttgctcatgcaaagcacaccagcagcaaacaatcgagccagaagagaactaccaaggtttatgaattatacactcaacaataccaaaaaaaccaatgtaacattaagcaagaaaaggaaaagaagatttcttggttttttgttaggtgttggatctgcaatcgccagtggcattgctgtatctaaggtcctgcacttagaaggagaagtgaacaagatcaaaagtgctctactatccacaaacaaggccgtagtcagcttatcaaatggagttagtgtcttaaccagcaaagtgttagacctcaaaaactatatagataaacaattgttacctattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacataatgtaaatgctggtaaatcaaccacaaatatcatgataactactataattatagtgattatagtaatattgttatcattaattgctgttggactgctcctatactgtaaggccagaagcacaccagtcacactaagcaaggatcaactgagtggtataaataatattgcatttagtaactga

SEQ ID NO: 10 - protein, 574 aa, Respiratory syncytial virus
MELPILKANAITTILAAVTFCFASSQNITEEFYQSTCSAVSKGYLSALRTGWYTSVITIELSNIKENKCNGTDAKVKLMKQELDKYKNAVTELQLLMQSTPAANNRARRELPRFMNYTLNNTKKTNVTLSKKRKRRFLGFLLGVGSAIASGIAVSKVLHLEGEVNKIKSALLSTNKAVVSLSNGVSVLTSKVLDLKNYIDKQLLPIVNKQSCRISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 11 - DNA, 897 bp, Respiratory syncytial virus
atgtccaaaaacaaggaccaacgcaccgctaagacactagaaaagacctgggacactctcaatcatttattattcatatcatcgggcttatataagttaaatcttaaatctatagcacaaatcacattatccattctggcaatgataatctcaacttcacttataattacagccatcatattcatagcctcggcaaaccacaaagtcacactaacaactgcaatcatacaagatgcaacaagccagatcaagaacacaaccccaacatacctcactcaggatcctcagcttggaatcagcttctccaatctgtctgaaattacatcacaaaccaccaccatactagcttcaacaacaccaggagtcaagtcaaacctgcaacccacaacagtcaagactaaaaacacaacaacaacccaaacacaacccagcaagcccactacaaaacaacgccaaaacaaaccaccaaacaaacccaataatgattttcacttcgaagtgtttaactttgtaccctgcagcatatgcagcaacaatccaacctgctgggctatctgcaaaagaataccaaacaaaaaaccaggaaagaaaaccaccaccaagcctacaaaaaaaccaaccttcaagacaaccaaaaaagatctcaaacctcaaaccactaaaccaaaggaagtacccaccaccaagcccacagaagagccaaccatcaacaccaccaaaacaaacatcacaactacactgctcaccaacaacaccacaggaaatccaaaactcacaagtcaaatggaaaccttccactcaacctcctccgaaggcaatctaagcccttctcaagtctccacaacatccgagcacccatcacaaccctcatctccacccaacacaacacgccagtag

SEQ ID NO: 12 - protein, 298 aa, Respiratory syncytial virus
MSKNKDQRTAKTLEKTWDTLNHLLFISSGLYKLNLKSIAQITLSILAMIISTSLIITAIIFIASANHKVTLTTAIIQDATSQIKNTTPTYLTQDPQLGISFSNLSEITSQTTTILASTTPGVKSNLQPTTVKTKNTTTTQTQPSKPTTKQRQNKPPNKPNNDFHFEVFNFVPCSICSNNPTCWAICKRIPNKKPGKKTTTKPTKKPTFKTTKKDLKPQTTKPKEVPTTKPTEEPTINTTKTNITTTLLTNNTTGNPKLTSQMETFHSTSSEGNLSPSQVSTTSEHPSQPSSPPNTTRQ

SEQ ID NO: 13 - DNA, 948 bp, Respiratory syncytial virus
atgtccaaaaacaagaatcaacgcactgccaggactctagaaaagacctgggatactcttaatcatctaattgtaatatcctcttgtttatacaaattaaatttaaaatctatagcacaaatagcactatcagttttggcaatgataatctcaacctctctcataattgcagccataatattcatcatctctgccaatcacaaagttacactaacaactgtcacagttcaaacaataaaaaaccacactgagaaaaacatcaccacttaccttactcaagtctcaccagaaagggttagcccatccaaacaacccacaaccacaccaccaatccacacaaactcagccacaatatcacctaatacaaaatcagaaacacaccatacaacagcacaaaccaaaggcagaaccaccactccaacacagaacaacaagccaagcacaaaaccacgtccaaaaaatccaccaaaaaaaccaaaagatgattaccattttgaagtattcaacttcgttccctgtagtatatgtggcaacaatcaactctgcaaatccatttgcaaaacaataccaagcaataaaccaaagaaaaaaccaaccataaaacccacaaacaaaccacccaccaaaaccacaaacaaaagagacccaaaaactctagccaaaacactgaaaaaagaaactaccatcaacccaacaaaaaaaccaacccccaagaccacagaaggagacaccagcacctcacagtccactgtgctcgacacaaccacatcaaagcacacagaaagagacaccagcacctcacaatccactgtgctcgacacaaccacatcaaaacacacaatccaacagcaatccctctactcaaccacccctgaaaacacacccaactccacacaaacacccacagcatccgagccctccacatcaaattccacccaaaaactctag

SEQ ID NO: 14 - protein, 315 aa, Respiratory syncytial virus
MSKNKNQRTARTLEKTWDTLNHLIVISSCLYKLNLKSIAQIALSVLAMIISTSLIIAAIIFIISANHKVTLTTVTVQTIKNHTEKNITTYLTQVSPERVSPSKQPTTTPPIHTNSATISPNTKSETHHTTAQTKGRTTTPTQNNKPSTKPRPKNPPKKPKDDYHFEVFNFVPCSICGNNQLCKSICKTIPSNKPKKKPTIKPTNKPPTKTTNKRDPKTLAKTLKKETTINPTKKPTPKTTEGDTSTSQSTVLDTTTSKHTERDTSTSQSTVLDTTTSKHTIQQQSLYSTTPENTPNSTQTPTASEPSTSNSTQKL

SEQ ID NO: 15 - DNA, 198 bp, Respiratory syncytial virus
atgggaaatacatccatcacaatagaattcacaagcaaattttggccctattttacactaatacatatgatcttaactctaatctctttactaattataatcactattatgattgcaatactaaataagctaagtgaacataaaacattctgtaacaatactcttgaactaggacagatgcatcaaatcaacacatag

SEQ ID NO: 16 - protein, 65 aa, Respiratory syncytial virus
MGNTSITIEFTSKFWPYFTLIHMILTLISLLIIITIMIAILNKLSEHKTFCNNTLELGQMHQINT

(19) United States
(12) Patent Application Publication
(10) Pub. No.: US 2011/0097358 A1
(43) Pub. Date: Apr. 28, 2011

(54) RESPIRATORY SYNCYTIAL VIRUS (RSV) VIRUS-LIKE PARTICLES (VLPs)

(75) Inventors: Jose M. Galarza, Scarsdale, NY (US); George R. Martin, Rockville, MD (US); Devyani Chaudhuri, Briarcliff Manor, NY (US); Andrew A. Fulvini, Yonkers, NY (US)

(73) Assignee: Techno Vax, Inc., Tarrytown, NY (US)

(21) Appl. No.: 12/902,733

(22) Filed: Oct. 12, 2010

Related U.S. Application Data
(60) Provisional application No. 61/250,783, filed on Oct. 12, 2009.
(60) Provisional application No. 61/250,791, filed on Oct. 12, 2009.

(51) Int. Cl. A61K 39/155; A61K 39/145; A61P 37/04; A61P 31/16; C12P 21/00

(52) U.S. Cl. 424/202.1; 424/211.1; 435/69.3

(57) ABSTRACT

Respiratory syncytial virus (RSV) virus-like particles (VLPs) comprising at least one RSV protein are described. Also described are compositions comprising these VLPs as well as methods of making and using these VLPs.

### CROSS REFERENCE TO RELATED APPLICATIONS

This application claims priority from U.S. Provisional Application Nos. 61/250,783 and 61/250,791, both filed Oct. 12, 2009, the disclosures of which are incorporated herein by reference.

### STATEMENT AS TO FEDERALLY SPONSORED RESEARCH

Not Applicable.

### TECHNICAL FIELD

Virus-like particles (VLPs) comprising respiratory syncytial (RVS) proteins are described, as are methods for making and using these VLPs.

### BACKGROUND

The respiratory syncytial virus has a single strand, negative-sense non segmented RNA genome and is classified as a member of the Pneumovirus genus within the Paramyxoviridae family. The viral genome is encapsidated by the N proteins which form the nucleocapsid which is associated with molecules of the L protein, the major polymerase component; the phosphoprotein P, a polymerase cofactor and the M2-1 protein. The ribnucleoproteins complex is packaged in a lipid enveloped derived from the host cell plasma membrane and acquired during morphogenesis and budding of the virus. The viral genome encodes at least eleven proteins comprising three transmembrane surface glycoproteins G, F and SH; three M proteins, the membrane associate matrix M, the M2-1 and the M2-2 both involve in the transcription/replication cycle; three nucleocapsid proteins (N, P, and L); and two nonstructural proteins (NS1 and NS2). See, Collins et al. (1994) J. Virol. 49:572-578); Collins and Crowe, 2007, Respiratory Syncytial Virus and Metapneumovirus, In Fields Virology, fifth ed, D. M. Knipe et al., (ed.). Lippincott Williams & Wilkins, Philadelphia, Pa.

Antigenic dimorphism between the subgroups of RSV A and B is mainly linked to the G protein, whereas the F protein is more closely related between the subgroups. The G and F proteins mediate attachment and entry of the virus into cells and syncytia formation. These surface proteins contain the antigenic determinants that elicit the partially protective antibody response by the host. Antigenic variations on the G protein are the major determinants that differentiate the two RSV subtypes, A and B. See, e.g., Connors et. al. (1991) J. Virol.; 65 (3):1634-7; Beeler et al. (1989) J. Virol.; 63(7):2941-50.) Both of these subtypes circulate in humans, probably with similar incidence and virulence. See, e.g., Tregoning et al. (2010) Clin. Microbiol. Rev. 23:74-98.

Respiratory syncytial virus is a significant causative agent of respiratory infections in infants, young children and the elderly as well as the general adult population. The World Health Organization (WHO) estimates that more than 3 million children younger than 5 die every year from lower respiratory tract infections (LRIs), with respiratory syncytial virus (RSV) infections accounting for more than 1 million deaths worldwide. In the United States, RSV infections cause about 4,500 deaths and result in the hospitalization of 125,000 infants every year with an estimated cost of approximately $900 million. In addition, to its significant morbidity during acute infection, RSV is associated with recurrent wheezing and respiratory abnormalities and research suggests that prior RSV infection is a significant risk factor for the development of asthma. See, e.g., Hall (1994) Science 265:1393-1394; Hall (1999) J. Pediatr., 135 (2): S2-S7.

The currently approved measures aimed at combating RSV include: 1) prophylaxis with a humanized anti-RSV-F monoclonal antibody administered by way of intramuscular injection each month from October to April, which plays an important role in the prevention of serious RSV infection in the highest risk infants, and 2) ribavirin, a partially effective drug with nonspecific antiviral activity including against RSV. An effective prophylactic vaccine capable of preventing RSV infection in any age group is not currently available. Therefore, development of a safe and effective RSV vaccine should have a great impact on the prevention and control of RSV disease, removing a significant public health burden, and reducing considerably the social and economical costs inflicted by this disease. See, e.g., Karron, In Vaccines Fifth Edition, Plotkin S A, Orenstein W A, Offit P A, Saunders Elsevier, 2008, pp 1283-1293.

Significant efforts, time and resources has been invested in research and development of an RSV vaccine, however in spite of all endeavors an effective RSV vaccine able to prevent infection with RSV and its associated disease is currently lacking. See, e.g., Dudas et al. (1998) Clin. Microbiol. Rev.; 11: 430-439. An early vaccine produced by formalin-inactivated virus failed to provide protection against RSV infection and some vaccinated individuals experienced an enhancement of disease subsequent to infection with the wild type virus (see, e.g., Kapikian et. al. (1969) Am. J. Epidemiol.; 89: 405-421; Kim et al. (1969), Am. J. Epidemiol.; 89: 422-434; Openshaw et al. (2005) Clin. Microbiol. Rev., 18: 541-555). Subsequent attempts have focused on developing live attenuated temperature-sensitive mutants generated by chemical mutagenesis or cold passages of wild type RSV or through recombinant methods (see, e.g., Collins et al. (1995) PNAS, 92:11563-11567; Gharpure et al. (1969) J. Virol., 3: 414-21; Crowe et al. (1994) Vaccine, 12: 783-90). Clinical evaluation of these vaccine in infants demonstrated that they were either over attenuated and unable to elicit a protective response or under attenuated causing symptoms typical of RSV infections (see, e.g., Kim et al. (1973) Pediatrics; 52: 56-63; Wright et al (1976) J. Pediatrics, 88: 931-936). Furthermore, some of the live attenuated vaccines were genetically unstable causing reversion to more infectious phenotypes (see, e.g., Hodes et al. (1974) Proc Soc Exp Biol Med.; 145:1158-64; Karron et al. (1997) J Infect Dis.; 176:1428-1436).

Thus, there remains a need for immunogenic RSV vaccine compositions and methods of making and using such compositions.

### SUMMARY

Described herein are virus-like particles (VLPs) comprising at least one RSV protein (e.g., antigens, structural proteins). Also described are compositions comprising these VLPs, as well as methods for making and using these VLPs. The VLPs described herein are devoid of viral genetic material and therefore unable to replicate or cause infection; however given their morphological, biochemical and antigenic similarities to wild type virions, VLPs are highly immunogenic and able to elicit robust protective immune responses. Unlike virion inactivated based vaccines, VLPs are not infectious eliminating the need for chemical treatment, thus maintaining the native conformation.

In one aspect, disclosed herein is a virus-like particle (VLP) comprising at least two matrix (M) proteins and an RSV F protein (wild-type, mutant and/or chimeric). In certain embodiments, at least one of the M proteins comprises an influenza matrix protein (M1 and/or M2). In other embodiments, the M protein comprises an influenza M1 protein or an RSV M protein and at least one other M protein comprises an influenza M2 protein or an RSV M2 protein. In embodiments in which the VLP does not contain an influenza matrix protein (e.g., VLPs including RSV M and M2 proteins and no influenza matrix proteins), the VLP further comprises an RSV SH protein. Furthermore, any of the VLPs described herein may further comprise one or more additional RSV proteins, for example one or more RSV-F proteins, one or more RSV G proteins, one or more RSV M (or M2) proteins and/or one or more RSV SH proteins.

The proteins in any of the VLPs described herein may be from any Group or strain of RSV or influenza virus, for example Group A or Group B RSV proteins. Proteins from different Groups can be present in the same VLP. In addition, the proteins of the VLP may be hybrid (or chimeric proteins) containing full-length or portions (wild-type or mutants) of viral proteins fused to full-length or portions (wild-type or mutants) of heterologous proteins. In certain embodiments, the hybrid proteins comprise RSV F and influenza amino acid sequences (e.g., the cytoplasmic tail and/or transmembrane domain of an influenza protein (e.g., HA) replaces the corresponding domain(s) in the RSV F protein). Furthermore, in any of the VLPs described herein, the proteins (e.g., hybrids) may contain one or more mutations with respect to wild-type proteins. In certain embodiments, the RSV F protein includes at least one modification that inhibits cleavage of the F protein (F0) into F1 and F2 and/or inhibits membrane fusion. In other embodiments, an RSV G protein of the VLP comprises a mutation in the central domain.

In another aspect, described herein is a host cell comprising any of the VLPs as described above. In certain embodiments, the host cell permits assembly and release of a VLP as described herein from one or more vectors encoding the polypeptides of the VLP. In certain embodiments, the eukaryotic cell is selected from the group consisting of a yeast cell, an insect cell, an amphibian cell, an avian cell, a plant cell or a mammalian cell.

In another aspect, described herein is a method of producing a VLP, the method comprising the steps of transfecting one or more expression vectors encoding two M proteins and an RSV F protein into a suitable host cell and expressing the combination of protein under conditions that allow VLP formation. In certain embodiments, at least one M protein comprises an influenza matrix protein. In other embodiments, additional or the same vectors may encode additional proteins, for example additional RSV proteins (G proteins, SH proteins, etc.). In other embodiments, at least one M protein comprises an RSV M protein. The expression vector may be a plasmid, a viral vector, a baculovirus vector or a non-viral vector. The vectors may encode one, more than one or all of the proteins of the VLP. In certain embodiments, one or more of the vectors are stably transfected into the host cell. In certain embodiments, the M proteins and F proteins are encoded on separate vectors and the vector encoding the M proteins is stably transfected into the cell prior to transfection with the vector encoding the RSV F protein. The proteins encoded by the vectors may be full-length wild-type, full-length mutants, truncated wild-type, truncated mutants (e.g., RSV G proteins comprising mutations in the central domain, RSV F proteins comprising modifications that inhibit cleavage into F1 and F2 and/or inhibit membrane fusion), and/or hybrid proteins include full-length and/or truncated wild-type or mutant proteins. The cell can be a eukaryotic cell, for example, a yeast cell, an insect cell, an amphibian cell, an avian cell, a plant cell or a mammalian cell.

In a still further aspect, described herein is an immunogenic composition comprising at least one VLP as described herein. In certain embodiments, the composition comprises and contains at least two VLPs, each VLP comprising a different RSV protein. In still further embodiments, the immunogenic composition further comprises an adjuvant.

In yet another aspect, described herein is a method of generating an immune response to RSV in a subject, the method comprising administering to the subject (e.g., human) an effective amount of one or more VLPs and/or immunogenic compositions as described herein. In certain embodiments, the composition is administered mucosally, intradermally, subcutaneously, intramuscularly and/or orally. In any of the methods described herein, the immune response generated can be sufficient to vaccinate the subject against RSV. Any of the methods may involve multiple administrations (e.g., a multiple dose schedule).

In another aspect, a packaging cell line is provided for producing RSV VLPs as described herein. The cell line is stably transfected with one or more polynucleotides encoding at least two M proteins and upon introduction and expression of the one or more RSV protein-encoding sequences not stably transfected into the cell, the VLP is produced by the cell. In certain embodiments, sequences encoding M1 and/or M2 are stably integrated into the packaging cell line and sequences encoding the RSV F protein (and optionally RSV G and/or SH proteins) expressed on the surface of the VLP are introduced into the cell such that the VLP is formed. In other embodiments, sequences encoding one or more of the RSV F proteins (and optionally RSV G and/or SH proteins) are stably integrated into the cell to form a packaging cell line and VLPs are formed upon introduction of sequences encoding the at least two M proteins. The packaging cell may be an insect, plant, mammalian, bacterial or fungal cell. In certain embodiments, the packaging cell is a mammalian (e.g., human) cell line.

### BRIEF DESCRIPTION OF THE DRAWINGS

FIG. 1, panels I to V, are schematics depicting exemplary DNA vectors carrying influenza and/or RSV genes within determined positions and under the control of the indicated regulatory elements.

FIG. 2 shows schematic diagrams of wild type RSV-F protein as well as exemplary hybrids and mutated forms of the RSV-F protein.

FIG. 3, panels A and B, depict examples of strategies employed for the transfection of vectors into mammalian cells, expression of proteins and selection of stably transfected cell lines for the continuous production of VLPs.

FIG. 4, panels A to C, show results of fluorescent activated cell sorting (FACS) of M1/M2 stably transfected mammalian cells using an anti-M2 antibody as primary and a fluorescein FITC labeled anti-mouse as secondary antibody. FIG. 4A shows unstained control. FIG. 4B shows isotype control and FIG. 4C shows staining for surface expression of M2.

FIG. 5, panels A and B, depict Western blots of cell lysates of M1/M2 stably transfected mammalian cells. FIG. 5A shows protein levels in MDCK cells (lane 1 shows results from normal MDCK cells and lane 2 shows results from MDCK cells constitutively expressing M1/M2 proteins. FIG. 5B shows results from CHO cells: lane 1 shows a negative control (CHO cell lysate); lane 2 shows CHO cells infected with influenza virus PR8; lane 3 shows CHO cells constitutively expressing M1/M2 (clone 1); and lane 4 shows CHO cells constitutively expressing M1/M2 (clone 2.)

FIG. 6, panels A and B, depict Western blot analysis of cell lysates, cell supernatants and concentrated supernatants of M1/M2 stably transfected MDCK cells (FIG. 6A) and CHO cells (FIG. 6B) which also were transfected with the RSV-F protein. The contents of each lane are indicated below each blot.

FIG. 7, panels A and B, depict Western blot analysis of cell lysates, cell supernatants and concentrated supernatants of M1/M2-F transfected Vero cells. FIG. 7A shows results when probing for M1 and F expression and FIG. 7B shows results when probing for RSV-F expression only. The contents of each lane are indicated below each blot.

FIG. 8, panels A to E, show electron micrograph images of flu/RSV hybrid VLPs purified from mammalian culture supernatant which were fixed with paraformaldehyde, stained with uranyl acetate and examined by electron microscopy. They show membrane surrounded particles that resemble an enveloped virus such as RSV.

FIG. 9 shows a cryo-electron micrograph image of flu/RSV hybrid VLP purified from mammalian culture supernatant as above.

### DETAILED DESCRIPTION

The practice of the present invention will employ, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); Fundamental Virology, Second Edition (Fields & Knipe eds., 1991, Raven Press, New York).

All publications, patents and patent applications cited herein are hereby incorporated by reference in their entirety.

As used in this specification and the appended claims, the singular forms “a,” “an” and “the” include plural references unless the content clearly dictates otherwise. Thus, for example, reference to “a VLP” includes a mixture of two or more such VLPs.

### DEFINITIONS

As used herein, the terms “sub-viral particle” “virus-like particle” or

“VLP” refer to a nonreplicating, viral shell. VLPs are generally composed of one or more viral proteins, such as, but not limited to those proteins referred to as capsid, coat, shell, surface and/or envelope proteins, or particle-forming polypeptides derived from these proteins. VLPs can form spontaneously upon recombinant expression of the protein in an appropriate expression system. Methods for producing particular VLPs are known in the art and discussed more fully below. The presence of VLPs following recombinant expression of viral proteins can be detected using conventional techniques known in the art, such as by electron microscopy, biophysical characterization, and the like. See, e.g., Baker et al., Biophys. J (1991) 60:1445-1456; Hagensee et al., J. Virol. (1994) 68:4503-4505. For example, VLPs can be isolated by density gradient centrifugation and/or identified by characteristic density banding (e.g., Examples). Alternatively, cryoelectron microscopy can be performed on vitrified aqueous samples of the VLP preparation in question, and images recorded under appropriate exposure conditions.

A used herein, the term “hybrid” or “chimeric” refers to a molecule (e.g., protein or VLP) that contains portions thereof, from at least two different proteins. For example, a hybrid RSV F protein refers to a protein comprising at least a portion of an RSV F protein (preferably a portion containing one or more antigenic determinants) and portions of a heterologous protein (e.g., the cytoplasmic and/or transmembrane domain of a different RSV F protein or a different viral protein, for example influenza HA or NA). It will be apparent that a hybrid molecule as described herein can include full-length proteins fused to additional heterologous polypeptides (full length or portions thereof) as well as portions proteins fused to additional heterologous polypeptides (full length or portions thereof). It will also be apparent that the hybrids can include wild-type sequences or mutant sequences in any one, some or all of the heterologous domains.

By “particle-forming polypeptide” derived from a particular viral protein is meant a full-length or near full-length viral protein, as well as a fragment thereof, or a viral protein with internal deletions, which has the ability to form VLPs under conditions that favor VLP formation. Accordingly, the polypeptide may comprise the full-length sequence, fragments, truncated and partial sequences, as well as analogs and precursor forms of the reference molecule. The term therefore intends deletions, additions and substitutions to the sequence, so long as the polypeptide retains the ability to form a VLP. Thus, the term includes natural variations of the specified polypeptide since variations in coat proteins often occur between viral isolates. The term also includes deletions, additions and substitutions that do not naturally occur in the reference protein, so long as the protein retains the ability to form a VLP. Preferred substitutions are those which are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids.

An “antigen” refers to a molecule containing one or more epitopes (either linear, conformational or both) that will stimulate a host's immune-system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 9, 10, 12 or 15 amino acids. The term includes polypeptides which include modifications, such as deletions, additions and substitutions (generally conservative in nature) as compared to a native sequence, so long as the protein maintains the ability to elicit an immunological response, as defined herein. These modifications may be deliberate, as through site-directed mutagenesis, or may be accidental, such as through mutations of hosts which produce the antigens.

An “immunological response” to an antigen or composition is the development in a subject of a humoral and/or a cellular immune response to an antigen present in the composition of interest. For purposes of the present disclosure, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. Hence, an immunological response may include one or more of the following effects: the production of antibodies by B-cells; and/or the activation of suppressor T-cells and/or γΔ T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

An “immunogenic composition” is a composition that comprises an antigenic molecule where administration of the composition to a subject results in the development in the subject of a humoral and/or a cellular immune response to the antigenic molecule of interest.

“Substantially purified” general refers to isolation of a substance (compound, polynucleotide, protein, polypeptide, polypeptide composition) such that the substance comprises the majority percent of the sample in which it resides. Typically in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence are determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

Typical “control elements”, include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences, and/or sequence elements controlling an open chromatin structure see e.g., McCaughan et al. (1995) PNAS USA 92:5431-5435; Kochetov et al (1998) FEBS Letts. 440:351-355.

A “nucleic acid” molecule can include, but is not limited to, prokaryotic sequences, eukaryotic mRNA, cDNA from eukaryotic mRNA, genomic DNA sequences from eukaryotic (e.g., mammalian) DNA, and even synthetic DNA sequences. The term also captures sequences that include any of the known base analogs of DNA and RNA.

“Operably linked” refers to an arrangement of elements wherein the components so described are configured so as to perform their usual function. Thus, a given promoter operably linked to a coding sequence is capable of effecting the expression of the coding sequence when active. The promoter need not be contiguous with the coding, sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Recombinant” as used herein to describe a nucleic acid molecule means a polynucleotide of genomic, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide. “Recombinant host cells,” “host cells,” “cells,” “cell lines,” “cell cultures,” and other such terms denoting prokaryotic microorganisms or eukaryotic cell lines cultured as unicellular entities, are used interchangeably, and refer to cells which can be, or have been, used as recipients for recombinant vectors or other transfer DNA, and include the progeny of the original cell which has been transfected. It is understood that the progeny of a single parental cell may not necessarily be completely identical in morphology or in genomic or total DNA complement to the original parent, due to accidental or deliberate mutation. Progeny of the parental cell which are sufficiently similar to the parent to be characterized by the relevant property, such as the presence of a nucleotide sequence encoding a desired peptide, are included in the progeny intended by this definition, and are covered by the above terms.

Techniques for determining amino acid sequence “similarity” are well known in the art. In general, “similarity” means the exact amino acid to amino acid comparison of two or more polypeptides at the appropriate place, where amino acids are identical or possess similar chemical and/or physical properties such as charge or hydrophobicity. A so-termed “percent similarity” then can be determined between the compared polypeptide sequences. Techniques for determining nucleic acid and amino acid sequence identity also are well known in the art and include determining the nucleotide sequence of the mRNA for that gene (usually via a cDNA intermediate) and determining the amino acid sequence encoded thereby, and comparing this to a second amino acid sequence. In general, “identity” refers to an exact nucleotide to nucleotide or amino acid to amino acid correspondence of two polynucleotides or polypeptide sequences, respectively.

Two or more polynucleotide sequences can be compared by determining their “percent identity.” Two or more amino acid sequences likewise can be compared by determining their “percent identity.” The percent identity of two sequences, whether nucleic acid or peptide sequences, is generally described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482-489 (1981). This algorithm can be extended to use with peptide sequences using the scoring matrix developed by Dayhoff, Atlas of Protein Sequences and Structure, M. O. Dayhoff ed., 5 suppl. 3:353-358, National Biomedical Research. Foundation, Washington, D.C., USA, and normalized by Gribskov, Nucl. Acids Res. 14(6):6745-6763 (1986). Suitable programs for calculating the percent identity or similarity between sequences are generally known in the art.

A “vector” is capable of transferring gene sequences to target cells (e.g., bacterial plasmid vectors, viral vectors, non-viral vectors, particulate carriers, and liposomes). Typically, “vector construct,” “expression vector,” and “gene transfer vector,” mean any nucleic acid construct capable of directing the expression of one or more sequences of interest in a host cell. Thus, the term includes cloning and expression vehicles, as well as viral vectors. The term is used interchangeable with the terms “nucleic acid expression vector” and “expression cassette.”

By “subject” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The system described above is intended for use in any of the above vertebrate species, since the immune systems of all of these vertebrates operate similarly.

By “pharmaceutically acceptable” or “pharmacologically acceptable” is meant a material which is not biologically or otherwise undesirable, i.e., the material may be administered to an individual in a formulation or composition without causing any unacceptable biological effects or interacting in a deleterious manner with any of the components of the composition in which it is contained.

As used herein, “treatment” refers to any of (i) the prevention of infection or reinfection, as in a traditional vaccine, (ii) the reduction or elimination of symptoms, and (iii) the substantial or complete elimination of the pathogen in question. Treatment may be effected prophylactically (prior to infection) or therapeutically (following infection).

As used herein the term “adjuvant” refers to a compound that, when used in combination with a specific immunogen (e.g. a VLP) in a formulation, will augment or otherwise alter or modify the resultant immune response. Modification of the immune response includes intensification or broadening the specificity of either or both antibody and cellular immune responses. Modification of the immune response can also mean decreasing or suppressing certain antigen-specific immune responses.

As used herein an “effective dose” generally refers to that amount of VLPs of the invention sufficient to induce immunity, to prevent and/or ameliorate an infection or to reduce at least one symptom of an infection and/or to enhance the efficacy of another dose of a VLP. An effective dose may refer to the amount of VLPs sufficient to delay or minimize the onset of an infection. An effective dose may also refer to the amount of VLPs that provides a therapeutic benefit in the treatment or management of an infection. Further, an effective dose is the amount with respect to VLPs of the invention alone, or in combination with other therapies, that provides a therapeutic benefit in the treatment or management of an infection. An effective dose may also be the amount sufficient to enhance a subject's (e.g., a human's) own immune response against a subsequent exposure to an infectious agent. Levels of immunity can be monitored, e.g., by measuring amounts of neutralizing secretory and/or serum antibodies, e.g., by plaque neutralization, complement fixation, enzyme-linked immunosorbent; or microneutralization assay. In the case of a vaccine, an “effective dose” is one that prevents disease and/or reduces the severity of symptoms.

As used herein, the term “effective amount” refers to an amount of VLPs necessary or sufficient to realize a desired biologic effect. An effective amount of the composition would be the amount that achieves a selected result, and such an amount could be determined as a matter of routine experimentation by a person skilled in the art. For example, an effective amount for preventing, treating and/or ameliorating an infection could be that amount necessary to cause activation of the immune system, resulting in the development of an antigen specific immune response upon exposure to VLPs of the invention. The term is also synonymous with “sufficient amount.”

As used herein, the term “multivalent” refers to VLPs which have multiple antigenic proteins against multiple types or strains of infectious agents.

As used herein the term “immune stimulator” refers to a compound that enhances an immune response via the body's own chemical messengers (cytokines). These molecules comprise various cytokines, lymphokines and chemokines with immunostimulatory, immunopotentiating, and pro-inflammatory activities, such as interferons, interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immune stimulator molecules can be administered in the same formulation as VLPs of the invention, or can be administered separately. Either the protein or an expression vector encoding the protein can be administered to produce an immunostimulatory effect.

As used herein the term “protective immune response” or “protective response” refers to an immune response mediated by antibodies against an infectious agent, which is exhibited by a vertebrate (e.g., a human), that prevents or ameliorates an infection or reduces at least one symptom thereof. VLPs of the invention can stimulate the production of antibodies that, for example, neutralize infectious agents, blocks infectious agents from entering cells, blocks replication of said infectious agents, and/or protect host cells from infection and destruction. The term can also refer to an immune response that is mediated by T-lymphocytes and/or other white blood cells against an infectious agent, exhibited by a vertebrate (e.g., a human), that prevents or ameliorates RSV infection or reduces at least one symptom thereof.

As use herein, the term “antigenic formulation” or “antigenic composition” refers to a preparation which, when administered to a vertebrate, e.g. a mammal, will induce an immune response.

As used herein, the term “vaccine” refers to a formulation which contains VLPs of the present invention, which is in a form that is capable of being administered to a vertebrate and which induces a protective immune response sufficient to induce immunity to prevent and/or ameliorate an infection and/or to reduce at least one symptom of an infection and/or to enhance the efficacy of another dose of VLPs. Typically, the vaccine comprises a conventional saline or buffered aqueous solution medium in which the composition of the present invention is suspended or dissolved. In this form, the composition of the present invention can be used conveniently to prevent, ameliorate, or otherwise treat an infection. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses.

### General Overview

Described herein are RSV VLPs that can be used to protect and/or treat humans from the RSV infection.

### Virus-Like Particles

When sequences encoding influenza proteins are expressed in eukaryotic, the proteins have been shown to self-assemble into noninfectious virus-like particles (VLP). See, Latham & Galarza (2001) J. Virol. 75(13):6154-6165; Galarza et al. (2005) Viral. Immunol. 18(1):244-51; and U.S. Patent Publications 2008/0233150; 2008/0031895 and 2009/0022762.

The present disclosure relates to RSV VLPs from the plasma membrane of eukaryotic cells, which VLPs carry on their surfaces an RSV protein, e.g., RSV-F protein. This VLP, alone or in combination with one or more adjuvants, stimulates an immune response that protects against RSV infection.

The RSV VLP (also called sub-viral structure vaccine (SVSV)) is composed of viral proteins produced from naturally occurring and/or mutated nucleic acid sequences of genes coding for matrix protein M (also known as M1) and, optionally, M2 protein. The matrix protein M is a universal component for the formation of all possible polyvalent sub-viral structure vaccine combinations. The M1 and M2 proteins may be derived from any virus. In certain embodiments, the M1 and/or M2 protein of the RSV VLP is derived from an influenza matrix protein. In other embodiments, the M1 and/or M2 protein of the RSV VLP is derived from RSV. The M1 and/or M2 proteins may be modified (mutated), for example as disclosed herein or in U.S. Patent Publications 2008/0031895 and 2009/0022762.

RSV proteins derived from the same or different families of enveloped viruses can be selected for incorporation onto the surface of the vaccine. The incorporation of RSV proteins into the same vaccine particle can be facilitated by replacing the cytoplasmic tail and transmembrane amino acid sequences with those from a common glycoprotein via alterations in the nucleic acids coding for these proteins. This approach allows for the design of a large number of possible polyvalent sub-viral vaccine combinations.

1. Polypeptide-Encoding Sequences

The VLPs produced as described herein are conveniently prepared using standard recombinant techniques. Polynucleotides encoding the RSV protein(s) and optionally influenza proteins are introduced into a host cell and, when the proteins are expressed in the cell, they assembly into VLPs.

Polynucleotide sequences coding for molecules (structural and/or antigen polypeptides) that form and/or incorporate into the VLPs can be obtained using recombinant methods, such as by screening cDNA and genomic libraries from cells expressing the gene, or by deriving the gene from a vector known to include the same. For example, plasmids which contain sequences that encode naturally occurring or altered cellular products may be obtained from a depository such as the A.T.C.C., or from commercial sources. Plasmids containing the nucleotide sequences of interest can be digested with appropriate restriction enzymes, and DNA fragments containing the nucleotide sequences can be inserted into a gene transfer vector using standard molecular biology techniques.

Alternatively, cDNA sequences may be obtained from cells which express or contain the sequences, using standard techniques, such as phenol extraction and PCR of cDNA or genomic DNA. See, e.g., Sambrook et al., supra, for a description of techniques used to obtain and isolate DNA. Briefly, mRNA from a cell which expresses the gene of interest can be reverse transcribed with reverse transcriptase using oligo-dT or random primers. The single stranded cDNA may then be amplified by PCR (see U.S. Pat. Nos. 4,683,202, 4,683,195 and 4,800,159, see also PCR Technology: Principles and Applications for DNA Amplification, Erlich (ed.), Stockton Press, 1989)) using oligonucleotide primers complementary-to sequences on either side of desired sequences.

The nucleotide sequence of interest can also be produced synthetically, rather than cloned, using a DNA synthesizer (e.g., an Applied Biosystems Model 392 DNA Synthesizer, available from ABI, Foster City, Calif.). The nucleotide sequence can be designed with the appropriate codons for the expression product desired. The complete sequence is assembled from overlapping oligonucleotides prepared by standard methods and assembled into a complete coding sequence. See, e.g., Edge (1981) Nature 292:756; Nambair et al. (1984) Science 223:1299; Jay et al. (1984) J. Biol. Chem. 259:6311.

The RSV VLPs described herein are typically formed by expressing sequences encoding M1, M2 and at least an RSV-F protein in a host cell. The expressed proteins self-assemble into VLPs with the antigenic glycoproteins decorating the surface of the VLP.

In certain embodiments, the matrix-encoding sequences are RSV matrix proteins. The nucleotide (SEQ ID NO:1) and amino acid (SEQ ID NO:2) sequence of an exemplary RSV matrix (M1 and M2) protein is shown below:

Nucleotide Sequence of RSV M (SEQ ID NO: 1)
ATGGAAACATACGTGAACAAGCTTCACGAAGGCTCCACATACACAGCT
GCTGTTCAATACAATGTCCTAGAAAAAGACGATGACCCTGCATCACTT
ACAATATGGGTGCCCATGTTCCAATCATCTATGCCAGCAGATTTACTT
ATAAAAGAACTAGCTAATGTCAACATACTAGTGAAACAAATATCCACA
CCCAAGGGACCTTCACTAAGAGTCATGATAAACTCAAGAAGTGCATTG
CTAGCACAAATGCCCAGCAAATTTACCATATGTGCTAATGTGTCCTTG
GATGAAAGAAGCAAACTGGCATATGATGTAACCACACCCTGTGAAATC
AAGGCATGTAGTCTAACATGCCTAAAATCAAAAAATATGTTAACTACA
GTTAAAGATCTCACTATGAAGACACTCAACCCCACACATGATATTATT
GCTTTATGTGAATTTGAAAACATAGTAACATCAAAAAAAGTCATAATA
CCAACATACCTAAGATCCATCAGTGTCAGAAATAAAGATCTGAACACA
CTTGAAAATATAACAACCACTGAATTCAAAAATGCCATCACAAATGCA
AAAATCATCCCTTACTCAGGATTACTATTAGTCATCACAGTGACTGAC
AACAAAGGAGCATTCAAATACATAAAGCCGCAAAGTCAATTCATAGTA
GATCTTGGAGCTTACCTAGAAAAAGAAAGTATATATTATGTTACCACA
AATTGGAAGCACACAGCTACACGATTTGCAATCAAACCCATGGAAGAT
TAA
Amino Acids Sequence RSV-M (SEQ ID NO: 2):
METYVNKLHEGSTYTAAVQYNVLEKDDDPASLTIWVPMFQSSMPADLL
IKELANVNILVKQISTPKGPSLRVMINSRSALLAQMPSKFTICANVSL
DERSKLAYDVTTPCEIKACSLTCLKSKNMLTTVKDLTMKTLNPTHDII
ALCEFENIVTSKKVIIPTYLRSISVRNKDLNTLENITTTEFKNAITNA
KIIPYSGLLLVITVTDNKGAFKYIKPQSQFIVDLGAYLEKESIYYVTT
NWKHTATRFAIKPMED

In other embodiments, the matrix-encoding sequences are influenza matrix proteins. The nucleotide (SEQ ID NO:3) and amino acid (SEQ ID NO:4) sequence of an exemplary influenza M1 protein and the nucleotide (SEQ ID NO:5) and amino acid (SEQ ID NO:6) sequence of an exemplary influenza M2 protein are shown below:

Nucleotide Sequence of Influenza M1
(SEQ ID NO: 3):
ATGAGCCTTCTGACCGAGGTCGAAACGTATGTTCTCTCTATCGTTCCG
TCAGGCCCCCTCAAAGCCGAGATCGCGCAGAGACTTGAAGATGTCTTT
GCTGGAAAGAACACAGATCTTGAGGCTCTCATGGAATGGCTAAAGACA
AGACCAATCCTGTCACCTCTGACTAAAGGGATTTTGGGATTTGTATTC
ACGCTCACCGTGCCCAGTGAGCGAGGACTGCAGCGTAGACGCTTTGTC
CAAAATGCCCTCAATGGGAATGGGGATCCAAATAACATGGACAGAGCA
GTTAAACTGTATAGAAAACTTAAGAGGGAGATAACATTCCATGGGGCC
AAAGAAATAGCACTCAGTTATTCTGCTGGTGCACTTGCCAGTTGCATG
GGCCTCATATACAACAGGATGGGGGCTGTGACCACTGAAGTGGCCTTT
GGCCTGGTTTGTGCAACCTGTGAACAGATTGCTGACTCCCAGCACAGG
TCTCATAGGCAAATGGTGGCAACAACCAATCCACTAATAAGACATGAG
AACAGAATGGTTCTGGCCAGCACTACAGCTAAGGCTATGGAGCAAATG
GCTGGATCAAGTGAGCAGGCAGCAGAGGCCATGGAGGTTGCTAGTCAG
GCCAGGCAAATGGTGCAGGCAATGAGAGCCATTGGGACTCATCCTAGC
TCCAGTGCTGGTCTAAAAGATGATCTTCTTGAAAATTTGCAGGCCTAT
CAGAAACGAATGGGGGTGCAGATGCAACGATTCAAGTGA
Amino Acids Sequence of Influenza M1
(SEQ ID NO: 4):
MSLLTEVETYVLSIVPSGPLKAEIAQRLEDVFAGKNTDLEALMEWLKT
RPILSPLTKGILGFVFTLTVPSERGLQRRRFVQNALNGNGDPNNMDRA
VKLYRKLKREITFHGAKEIALSYSAGALASCMGLIYNRMGAVTTEVAF
GLVCATCEQIADSQHRSHRQMVATTNPLIRHENRMVLASTTAKAMEQM
AGSSEQAAEAMEVASQARQMVQAMRAIGTHPSSSAGLKDDLLENLQAY
QKRMGVQMQRFK
Nucleotide Sequence of Influenza M2
(SEQ ID NO: 5):
ATGAGCCTTCTGACCGAGGTCGAAACGCCTATCAGAAACGAATGGGGG
TGCAGATGCAACGATTCAAGTGACCCTCTTGTTGTTGCTGCGAGTATC
ATTGGGATCTTGCACTTGATATTGTGGATTCTTGATCGTCTTTTTTTC
AAATGCATCTATCGATTCTTTGAACACGGTCTGAAAAGAGGGCCTTCT
ACGGAAGGAGTACCTGAGTCTATGAGGGAAGAATATCGAAAGGAACAG
CAGAGTGCTGTGGATGCTGACGACAGTCATTTTGTCAGCATAGAGCTG
GAGTAA
Amino Acids Sequence of Influenza M2
(SEQ ID NO: 6):
MSLLTEVETPIRNEWGCRCNDSSDPLVVAASIIGILHLILWILDRLFF
KCIYRFFEHGLKRGPSTEGVPESMREEYRKEQQSAVDADDSHFVSIE
LE

It will also be apparent that the matrix-encoding sequences can contain one or more mutations, for example as described in U.S. Patent Publications 2008/0031895 and 2009/0022762.

In addition, the RSV VLPs as described herein will also typically include at least one RSV F protein, either wild-type or mutant (and/or a hybrid of wild-type or mutant with another viral protein or portions thereof). Along with RSV G, RSV F proteins are primarily responsible for viral recognition and entry into target cells; G protein binds to a specific cellular receptor and the F protein promotes fusion of the virus with the cell. The F protein is also expressed on the surface of infected cells and is responsible for subsequent fusion with other cells leading to syncytia formation. Thus, antibodies to the F protein can neutralize virus or block entry of the virus into the cell or prevent syncytia formation. Although antigenic and structural differences between A and B subtypes have been described for both the G and F proteins, the more significant antigenic differences reside on the G protein, where amino acid sequences are only 53% homologous and antigenic relatedness is 5% (Walsh et al. (1987) J. Infect. Dis. 155: 1198-1204; and Johnson et al. (1987) Proc. Natl. Acad. Sci. USA 84, 5625-5629). Conversely, antibodies raised to the F protein show a high degree of cross-reactivity among subtype A and B viruses.

The RSV F protein directs penetration of RSV by fusion between the virion's envelope protein and the host cell plasma membrane. Later in infection, the F protein expressed on the cell surface can mediate fusion with neighboring cells to form syncytia. The F protein is a type I transmembrane surface protein that has a N-terminal cleaved signal peptide and a membrane anchor near the C-terminus. RSV F is synthesized as an inactive F0 precursor that assembles into a homotrimer and is activated by cleavage in the trans-Golgi complex by a cellular endoprotease to yield two disulfide-linked subunits. The N-terminus of the F1 subunit that is created by cleavage contains a hydrophobic domain (the fusion peptide) that inserts directly into the target membrane to initiate fusion. The F1 subunit also contains heptad repeats that associate during fusion, driving a conformational shift that brings the viral and cellular membranes into close proximity (Collins and Crowe, 2007, Fields Virology, 5th ed., D. M. Knipe et al., Lippincott, Williams and Wilkons, p. 1604).

Sequences of exemplary RSV F proteins are shown below. In the exemplary mutants, mutations are shown in bold, underlining and/or italics and the positions mutated are indicated below the sequence.

```
Nucleotide Sequence of RSV F (Wild Type)
(SEQ ID NO: 7):
ATGGAGTTGCCAATCCTCAAAGCAAATGCAATTACCACAATCCTCGCT
GCAGTCACATTTTGCTTTGCTTCTAGTCAAAACATCACTGAAGAATTT
TATCAATCAACATGCAGTGCAGTTAGCAAAGGCTATCTTAGTGCTCTA
AGAACTGGTTGGTATACTAGTGTTATAACTATAGAATTAAGTAATATC
AAGGAAAATAAGTGTAATGGAACAGATGCTAAGGTAAAATTGATAAAC
CAAGAATTAGATAAATATAAAAATGCTGTAACAGAATTGCAGTTGCTC
ATGCAAAGCACAACAGCAGCAAACAATCGAGCCAGAAGAGAACTACCA
AGGTTTATGAATTATACACTCAACAATACCAAAAAAACCAATGTAACA
TTAAGCAAGAAAAGGAAAAGAAGATTTCTTGGTTTTTTGTTAGGTGTT
GGATCTGCAATCGCCAGTGGCATTGCTGTATCTAAGGTCCTGCACTTA
GAAGGAGAAGTGAACAAGATCAAAAGTGCTCTACTATCCACAAACAAG
GCCGTAGTCAGCTTATCAAATGGAGTTAGTGTCTTAACCAGCAAAGTG
TTAGACCTCAAAAACTATATAGATAAACAATTGTTACCTATTGTGAAT
AAGCAAAGCTGCAGAATATCAAATATAGAAACTGTGATAGAGTTCCAA
CAAAAGAACAACAGACTACTAGAGATTACCAGGGAATTTAGTGTTAAT
GCAGGTGTAACTACACCTGTAAGCACTTACATGTTAACTAATAGTGAA
TTATTGTCATTAATCAATGATATGCCTATAACAAATGATCAGAAAAAG
TTAATGTCCAACAATGTTCAAATAGTTAGACAGCAAAGTTACTCTATC
ATGTCCATAATAAAAGAGGAAGTCTTAGCATATGTAGTACAATTACCA
CTATATGGTGTGATAGATACACCTTGTTGGAAATTACACACATCCCCT
CTATGTACAACCAACACAAAAGAAGGGTCAAACATCTGTTTAACAAGA
ACTGACAGAGGATGGTACTGTGACAATGCAGGATCAGTATCTTTCTTC
CCACAAGCTGAAACATGTAAAGTTCAATCGAATCGAGTATTTTGTGAC
ACAATGAACAGTTTAACATTACCAAGTGAAGTAAATCTCTGCAATGTT
GACATATTCAATCCCAAATATGATTGTAAAATTATGACTTCAAAAACA
GATGTAAGCAGCTCCGTTATCACATCTCTAGGAGCCATTGTGTCATGC
TATGGCAAAACTAAATGTACAGCATCCAATAAAAATCGTGGAATCATA
AAGACATTTTCTAACGGGTGTGATTATGTATCAAATAAAGGGGTGGAC
ACTGTGTCTGTAGGTAACACATTATATTATGTAAATAAGCAAGAAGGC
AAAAGTCTCTATGTAAAAGGTGAACCAATAATAAATTTCTATGACCCA
TTAGTATTCCCCTCTGATGAATTTGATGCATCAATATCTCAAGTCAAT
GAGAAGATTAACCAGAGTTTAGCATTTATTCGTAAATCCGATGAATTA
TTACATCATGTAAATGCTGGTAAATCAACCACAAATATCATGATAACT
ACTATAATTATAGTGATTATAGTAATATTGTTATCATTAATTGCTGTT
GGACTGCTCCTATACTGTAAGGCCAGAAGCACACCAGTCACACTAAGC
AAGGATCAACTGAGTGGTATAAATAATATTGCATTTAGTAACTGA
Amino Acids Sequence of RSV F (wild type)
(SEQ ID NO: 8)
MELPILKANAITTILAAVTFCFASSQNITEEFYQSTCSAVSKGYLSAL
RTGWYTSVITIELSNIKENKCNGTDAKVKLINQELDKYKNAVTELQLL
MQSTTAANNRARRELPRFMNYTLNNTKKTNVTLSKKRKRRFLGFLLGV
GSAIASGIAVSKVLHLEGEVNKIKSALLSTNKAVVSLSNGVSVLTSKV
LDLKNYIDKQLLPIVNKQSCRISNIETVIEFQQKNNRLLEITREFSVN
AGVTTPVSTYMLTNSELLSLINDMPITNDQKKLMSNNVQIVRQQSYSI
MSIIKEEVLAYVVQLPLYGVIDTPCWKLHTSPLCTTNTKEGSNICLTR
TDRGWYCDNAGSVSFFPQAETCKVQSNRVFCDTMNSLTLPSEVNLCNV
DIFNPKYDCKIMTSKTDVSSSVITSLGAIVSCYGKTKCTASNKNRGII
KTFSNGCDYVSNKGVDTVSVGNTLYYVNKQEGKSLYVKGEPIINFYDP
LVFPSDEFDASISQVNEKINQSLAFIRKSDELLHHVNAGKSTTNIMIT
TIIIVIIVILLSLIAVGLLLYCKARSTPVTLSKDQLSGINNIAFSN
Nucleotide Sequence of RSV F (Mutant #1)
(SEQ ID NO: 9):
ATGGAGTTGCCAATCCTCAAAGCAAATGCAATTACCACAATCCTCGCT
GCAGTCACATTTTGCTTTGCTTCTAGTCAAAACATCACTGAAGAATTT
TATCAATCAACATGCAGTGCAGTTAGCAAAGGCTATCTTAGTGCTCTA
AGAACTGGTTGGTATACTAGTGTTATAACTATAGAATTAAGTAATATC
AAGGAAAATAAGTGTAATGGAACAGATGCTAAGGTAAAATTGAT [symbol] AAA
_CAAGAATTAGATAAATATAAAAATGCTGTAACAGAATTGCAGTTGCT
CATGCAAAGCACA [symbol] CAGCAGCAAACAATCGAGCCAGAAGAGAACTACC
AAGGTTTATGAATTATACACTCAACAATACCAAAAAAACCAATGTAAC
ATTAAGCAAGAAAAGGAAAAGAAGATTTCTTGGTTTTTTGTTAGGTGT
TGGATCTGCAATCGCCAGTGGCATTGCTGTATCTAAGGTCCTGCACTT
AGAAGGAGAAGTGAACAAGATCAAAAGTGCTCTACTATCCACAAACAA
GGCCGTAGTCAGCTTATCAAATGGAGTTAGTGTCTTAACCAGCAAAGT
GTTAGACCTCAAAAACTATATAGATAAACAATTGTTACCTATTGTGAA
TAAGCAAAGCTGCAGAATATCAAATATAGAAACTGTGATAGAGTTCCA
ACAAAAGAACAACAGACTACTAGAGATTACCAGGGAATTTAGTGTTAA
TGCAGGTGTAACTACACCTGTAAGCACTTACATGTTAACTAATAGTGA
ATTATTGTCATTAATCAATGATATGCCTATAACAAATGATCAGAAAAA
GTTAATGTCCAACAATGTTCAAATAGTTAGACAGCAAAGTTACTCTAT
CATGTCCATAATAAAAGAGGAAGTCTTAGCATATGTAGTACAATTACC
ACTATATGGTGTGATAGATACACCTTGTTGGAAATTACACACATCCCC
TCTATGTACAACCAACACAAAAGAAGGGTCAAACATCTGTTTAACAAG
AACTGACAGAGGATGGTACTGTGACAATGCAGGATCAGTATCTTTCTT
CCCACAAGCTGAAACATGTAAAGTTCAATCGAATCGAGTATTTTGTGA
CACAATGAACAGTTTAACATTACCAAGTGAAGTAAATCTCTGCAATGT
TGACATATTCAATCCCAAATATGATTGTAAAATTATGACTTCAAAAAC
AGATGTAAGCAGCTCCGTTATCACATCTCTAGGAGCCATTGTGTCATG
CTATGGCAAAACTAAATGTACAGCATCCAATAAAAATCGTGGAATCAT
AAAGACATTTTCTAACGGGTGTGATTATGTATCAAATAAAGGGGTGGA
CACTGTGTCTGTAGGTAACACATTATATTATGTAAATAAGCAAGAAGG
CAAAAGTCTCTATGTAAAAGGTGAACCAATAATAAATTTCTATGACCC
ATTAGTATTCCCCTCTGATGAATTTGATGCATCAATATCTCAAGTCAA
TGAGAAGATTAACCAGAGTTTAGCATTTATTCGTAAATCCGATGAATT
ATTACAT [symbol] ATGTAAATGCTGGTAAATCAACCACAAATATCATGATAAC
TACTATAATTATAGTGATTATAGTAATATTGTTATCATTAATTGCTGT
TGGACTGCTCCTATACTGTAAGGCCAGAAGCACACCAGTCACACTAAG
CAAGGATCAACTGAGTGGTATAAATAATATTGCATTTAGTAACTGA
Mutations in mutant #1 as compared to wild-type:
Insertion: 236 G
Deletion: 240 C
Mutations: 301 A→C
                    1543 C→A
Amino Acids Sequence of RSV-F (Mutant #2)
(SEQ ID NO: 10):
MELPILKANAITTILAAVTFCFASSQNITEEFYQSTCSAVSKGYLSAL
RTGWYTSVITIELSNIKENKCNGTDAKVKL [symbol] QELDKYKNAVTELQLL
MQST [symbol] AANNRARRELPRFMNYTLNNTKKTNVTLSKKRKRRFLGFLLGV
GSAIASGIAVSKVLHLEGEVNKIKSALLSTNKAVVSLSNGVSVLTSKV
LDLKNYIDKQLLPIVNKQSCRISNIETVIEFQQKNNRLLEITREFSVN
AGVTTPVSTYMLTNSELLSLINDMPITNDQKKLMSNNVQIVRQQSYSI
MSIIKEEVLAYVVQLPLYGVIDTPCWKLHTSPLCTTNTKEGSNICLTR
TDRGWYCDNAGSVSFFPQAETCKVQSNRVFCDTMNSLTLPSEVNLCNV
DIFNPKYDCKIMTSKTDVSSSVITSLGAIVSCYGKTKCTASNKNRGII
KTFSNGCDYVSNKGVDTVSVGNTLYYVNKQEGKSLYVKGEPIINFYDP
LVFPSDEFDASISQVNEKINQSLAFIRKSDELLH [symbol] VNAGKSTTNIMIT
TIIIVIIVILLSLIAVGLLLYCKARSTPVTLSKDQLSGINNIAFSN
Mutations in mutant #2 as compared to wild-type:
  79 (I→M)
  80 (N→K)
101 (T→P)
515 (H→N)
```

The VLPs described herein may further comprise additional RSV and/or influenza proteins. In certain embodiments, the VLPs comprise one or more RSV-GA proteins (wild-type, mutant and/or hybrids of wild-type or mutants), one or more RSV-GB proteins (wild-type, mutant and/or hybrids of wild-type or mutants) and/or one or more RSV SH proteins (wild-type, mutant and/or hybrids of wild-type or mutants). Exemplary additional RSV protein nucleotide and amino acid sequences are shown below.

Nucleotide Sequence of RSV GA (SEQ ID NO: 11):
ATGTCCAAAAACAAGGACCAACGCACCGCTAAGACACTAGAAAAGACC
TGGGACACTCTCAATCATTTATTATTCATATCATCGGGCTTATATAAG
TTAAATCTTAAATCTATAGCACAAATCACATTATCCATTCTGGCAATG
ATAATCTCAACTTCACTTATAATTACAGCCATCATATTCATAGCCTCG
GCAAACCACAAAGTCACACTAACAACTGCAATCATACAAGATGCAACA
AGCCAGATCAAGAACACAACCCCAACATACCTCACTCAGGATCCTCAG
CTTGGAATCAGCTTCTCCAATCTGTCTGAAATTACATCACAAACCACC
ACCATACTAGCTTCAACAACACCAGGAGTCAAGTCAAACCTGCAACCC
ACAACAGTCAAGACTAAAAACACAACAACAACCCAAACACAACCCAGC
AAGCCCACTACAAAACAACGCCAAAACAAACCACCAAACAAACCCAAT
AATGATTTTCACTTCGAAGTGTTTAACTTTGTACCCTGCAGCATATGC
AGCAACAATCCAACCTGCTGGGCTATCTGCAAAAGAATACCAAACAAA
AAACCAGGAAAGAAAACCACCACCAAGCCTACAAAAAAACCAACCTTC
AAGACAACCAAAAAAGATCTCAAACCTCAAACCACTAAACCAAAGGAA
GTACCCACCACCAAGCCCACAGAAGAGCCAACCATCAACACCACCAAA
ACAAACATCACAACTACACTGCTCACCAACAACACCACAGGAAATCCA
AAACTCACAAGTCAAATGGAAACCTTCCACTCAACCTCCTCCGAAGGC
AATCTAAGCCCTTCTCAAGTCTCCACAACATCCGAGCACCCATCACAA
CCCTCATCTCCACCCAACACAACACGCCAGTAG
Amino Acids Sequence RSV-GA (SEQ ID NO: 12):
MSKNKDQRTAKTLEKTWDTLNHLLFISSGLYKLNLKSIAQITLSILAM
IISTSLIITAIIFIASANHKVTLTTAIIQDATSQIKNTTPTYLTQDPQ
LGISFSNLSEITSQTTTILASTTPGVKSNLQPTTVKTKNTTTTQTQPS
KPTTKQRQNKPPNKPNNDFHFEVFNEVPCSICSNNPTCWAICKRIPNK
KPGKKTTTKPTKKPTFKTTKKDLKPQTTKPKEVPTTKPTEEPTINTTK
TNITTTLLTNNTTGNPKLTSQMETFHSTSSEGNLSPSQVSTTSEHPSQ
PSSPPNTTRQ
Nucleotide Sequence of RSV GB (SEQ ID NO: 13):
ATGTCCAAAAACAAGAATCAACGCACTGCCAGGACTCTAGAAAAGACC
TGGGATACTCTTAATCATCTAATTGTAATATCCTCTTGTTTATACAAA
TTAAATTTAAAATCTATAGCACAAATAGCACTATCAGTTTTGGCAATG
ATAATCTCAACCTCTCTCATAATTGCAGCCATAATATTCATCATCTCT
GCCAATCACAAAGTTACACTAACAACTGTCACAGTTCAAACAATAAAA
AACCACACTGAGAAAAACATCACCACTTACCTTACTCAAGTCTCACCA
GAAAGGGTTAGCCCATCCAAACAACCCACAACCACACCACCAATCCAC
ACAAACTCAGCCACAATATCACCTAATACAAAATCAGAAACACACCAT
ACAACAGCACAAACCAAAGGCAGAACCACCACTCCAACACAGAACAAC
AAGCCAAGCACAAAACCACGTCCAAAAAATCCACCAAAAAAACCAAAA
GATGATTACCATTTTGAAGTATTCAACTTCGTTCCCTGTAGTATATGT
GGCAACAATCAACTCTGCAAATCCATTTGCAAAACAATACCAAGCAAT
AAACCAAAGAAAAAACCAACCATAAAACCCACAAACAAACCACCCACC
AAAACCACAAACAAAAGAGACCCAAAAACTCTAGCCAAAACACTGAAA
AAAGAAACTACCATCAACCCAACAAAAAAACCAACCCCCAAGACCACA
GAAGGAGACACCAGCACCTCACAGTCCACTGTGCTCGACACAACCACA
TCAAAGCACACAGAAAGAGACACCAGCACCTCACAATCCACTGTGCTC
GACACAACCACATCAAAACACACAATCCAACAGCAATCCCTCTACTCA
ACCACCCCTGAAAACACACCCAACTCCACACAAACACCCACAGCATCC
GAGCCCTCCACATCAAATTCCACCCAAAAACTCTAG
Amino Acids Sequence RSV-GA (SEQ ID NO: 14):
MSKNKNQRTARTLEKTWDTLNHLIVISSCLYKLNLKSIAQIALSVLAM
IISTSLIIAAIIFIISANHKVTLTTVTVQTIKNHTEKNITTYLTQVSP
ERVSPSKQPTTTPPIHTNSATISPNTKSETHHTTAQTKGRTTTPTQNN
KPSTKPRPKNPPKKPKDDYHFEVFNFVPCSICGNNQLCKSICKTIPSN
KPKKKPTIKPTNKPPTKTTNKRDPKTLAKTLKKETTINPTKKPTPKTT
EGDTSTSQSTVLDTTTSKHTERDTSTSQSTVLDTTTSKHTIQQQSLYS
TTPENTPNSTQTPTASEPSTSNSTQKL
Nucleotide Sequence of RSV SH (SEQ ID NO: 15):
ATGGGAAATACATCCATCACAATAGAATTCACAAGCAAATTTTGGCCC
TATTTTACACTAATACATATGATCTTAACTCTAATCTCTTTACTAATT
ATAATCACTATTATGATTGCAATACTAAATAAGCTAAGTGAACATAAA
ACATTCTGTAACAATACTCTTGAACTAGGACAGATGCATCAAATCAAC
ACATAG
Amino Acids Sequence RSV-SH (SEQ ID NO: 16):
MGNTSITIEFTSKFWPYFTLIHMILTLISLLIIITIMIAILNKLSEHK
TFCNNTLELGQMHQINT

Any of the proteins used in the RSV VLPs described herein may be hybrid (or chimeric) proteins. It will be apparent that all or parts of the polypeptides may be replaced with sequences from other viruses and/or sequences from other influenza strains. In one exemplary embodiment, sequences encoding the RSV F protein are hybrids in that they include heterologous sequences encoding the transmembrane and/or cytoplasmic tail domains, for example domains from influenza proteins such as HA or NA. See, e.g., U.S. Patent Publication Nos. 2008/0031895 and 2009/0022762.

Preferably, the RSV and/or influenza sequences employed to form influenza VLPs exhibit between about 60% to 80% (or any value therebetween including 61%, 62%, 63%, 64%, 65%, 66%, 67%, 68%, 69%, 70%, 71%, 72%, 73%, 74%, 75%, 76%, 77%, 78% and 79%) sequence identity to a naturally occurring RSV and influenza polynucleotide sequence and more preferably the sequences exhibit between about 80% and 100% (or any value therebetween including 81%, 82%, 83%, 84%, 85%, 86%, 87%, 88%, 89%, 90%, 91%, 92%, 93%, 94%, 95%, 96%, 97%, 98% and 99%) sequence identity to a naturally occurring RSV or influenza polynucleotide sequence.

Any of the sequences described herein may further include additional sequences. For example, to further to enhance vaccine potency, hybrid molecules are expressed and incorporated into the sub-viral structure. These hybrid molecules are generated by linking, at the DNA level, the sequences coding for the matrix protein genes with sequences coding for an adjuvant or immuno-regulatory moiety. During sub-viral structure formation, these hybrid proteins are incorporated into or onto the particle depending on whether M1 or optional M2 carries the adjuvant molecule. The incorporation of one or more polypeptide immunomodulatory polypeptides (e.g., adjuvants describe in detail below) into the sequences described herein into the VLP may enhance potency and therefore reduces the amount of antigen required for stimulating a protective immune response. Alternatively, as described below, one or more additional molecules (polypeptide or small molecules) may be included in the VLP-containing compositions after production of the VLP from the sequences described herein.

These sub-viral structures do not contain infectious viral nucleic acids and they are not infectious eliminating the need for chemical inactivation. Absence of chemical treatment preserves native epitopes and protein conformations enhancing the immunogenic characteristics of the vaccine.

The sequences described herein can be operably linked to each other in any combination. For example, one or more sequences may be expressed from the same promoter and/or from different promoters. As described below, sequences may be included on one or more vectors.

2. Expression Vectors

Once the constructs comprising the sequences encoding the RSV polypeptide(s) desired to be incorporated into the VLP have been synthesized, they can be cloned into any suitable vector or replicon for expression. Numerous cloning vectors are known to those of skill in the art, and one having ordinary skill in the art can readily select appropriate vectors and control elements for any given host cell type in view of the teachings of the present specification and information known in the art about expression. See, generally, Ausubel et al, supra or Sambrook et al, supra.

Non-limiting examples of vectors that can be used to express sequences that assembly into VLPs as described herein include viral-based vectors (e.g., retrovirus, adenovirus, adeno-associated virus, lentivirus), baculovirus vectors (see, Examples), plasmid vectors, non-viral vectors, mammalians vectors, mammalian artificial chromosomes (e.g., liposomes, particulate carriers, etc.) and combinations thereof.

The expression vector(s) typically contain(s) coding sequences and expression control elements which allow expression of the coding regions in a suitable host. The control elements generally include a promoter, translation initiation codon, and translation and transcription termination sequences, and an insertion site for introducing the insert into the vector. Translational control elements have been reviewed by M. Kozak (e.g., Kozak, M., Mamm. Genome 7(8):563-574, 1996; Kozak, M., Biochimie 76(9):815-821, 1994; Kozak, M., J Cell Biol 108(2):229-241, 1989; Kozak, M., and Shatkin, A. J., Methods Enzymol 60:360-375, 1979).

For example, typical promoters for mammalian cell expression include the SV40 early promoter, a CMV promoter such as the CMV immediate early promoter (a CMV promoter can include intron A), RSV, HIV-LTR, the mouse mammary tumor virus LTR promoter (MMLV-LTR), FIV-LTR, the adenovirus major late promoter (Ad MLP), and the herpes simplex virus promoter, among others. Other nonviral promoters, such as a promoter derived from the murine metallothionein gene, will also find use for mammalian expression. Typically, transcription termination and polyadenylation sequences will also be present, located 3′ to the translation stop codon. Preferably, a sequence for optimization of initiation of translation, located 5′ to the coding sequence, is also present. Examples of transcription terminator/polyadenylation signals include those derived from SV40, as described in Sambrook, et al., supra, as well as a bovine growth hormone terminator sequence. Introns, containing splice donor and acceptor sites, may also be designed into the constructs as described herein (Chapman et al., Nuc. Acids Res. (1991) 19:3979-3986).

Enhancer elements may also be used herein to increase expression levels of the mammalian constructs. Examples include the SV40 early gene enhancer, as described in Dijkema et al., EMBO J. (1985) 4:761, the enhancer/promoter derived from the long terminal repeat (LTR) of the Rous Sarcoma Virus, as described in Gorman et al., Proc. Natl. Acad. Sci. USA (1982b) 79:6777 and elements derived from human CMV, as described in Boshart et al., Cell (1985) 41:521, such as elements included in the CMV intron A sequence (Chapman et al., Nuc. Acids Res. (1991) 19:3979-3986).

It will be apparent that one or more vectors may contain one or more sequences encoding proteins to be incorporated into the VLP. For example, a single vector may carry sequences encoding all the proteins found in the VLP. Alternatively, multiple vectors may be used (e.g., multiple constructs, each encoding a single polypeptide-encoding sequence or multiple constructs, each encoding one or more polypeptide-encoding sequences). In embodiments in which a single vector comprises multiple polypeptide-encoding sequences, the sequences may be operably linked to the same or different transcriptional control elements (e.g., promoters) within the same vector. Furthermore, vectors may contain additional gene expression controlling sequences including chromatin opening elements which prevent transgene silencing and confer consistent, stable and high level of gene expression, irrespective of the chromosomal integration site. These are DNA sequence motifs located in proximity of house-keeping genes, which in the vectors create a transcriptionally active open chromatin environment around the integrated transgene, maximizing transcription and protein expression, irrespective of the position of the transgene in the chromosome.

In addition, one or more sequences encoding non-influenza proteins may be expressed and incorporated into the VLP, including, but not limited to, sequences comprising and/or encoding immunomodulatory molecules (e.g., adjuvants described below), for example, immunomodulating oligonucleotides (e.g., CpGs), cytokines, detoxified bacterial toxins and the like.

3. VLP Production

As noted above, influenza proteins expressed in a eukaryotic host cell have been shown to self-assemble into noninfectious virus-like particles (VLP). Accordingly, the sequences and/or vectors described herein are then used to transform an appropriate host cell. The construct(s) encoding the proteins that form the VLPs described herein provide efficient means for the production of influenza VLPs using a variety of different cell types, including, but not limited to, insect, fungal (yeast) and mammalian cells.

Preferably, the sub-viral structure vaccines are produced in eukaryotic cells following transfection, establishment of continuous cell lines (using standard protocols) and/or infection with DNA constructs that carry the influenza genes of interest as known to one skilled in the art. The level of expression of the proteins required for sub-viral structure formation is maximized by sequence optimization of the eukaryotic or viral promoters that drive transcription of the selected genes. The sub-viral structure vaccine is released into the culture media, from where it is purified and subsequently formulated as a vaccine. The sub-viral structures are not infectious and therefore inactivation of the VLP is not required as it is for some killed viral vaccines

The ability of influenza polypeptides expressed from sequences as described herein to self-assemble into VLPs with antigenic glycoproteins presented on the surface allows these VLPs to be produced in many host cell by co-introduction of the desired sequences. The sequence(s) (e.g., in one or more expression vectors) may be stably and/or transiently integrated in various combinations into a host cell.

Suitable host cells include, but are not limited to, bacterial, mammalian, baculovirus/insect, yeast, plant and Xenopus cells.

For example, a number of mammalian cell lines are known in the art and include primary cells as well as immortalized cell lines available from the American Type Culture Collection (A.T.C.C.), such as, but not limited to, MDCK, BHK, VERO, MRC-5, WI-38, HT1080, 293, 293T, RD, COS-7, CHO, Jurkat, HUT, SUPT, C8166, MOLT4/clone8, MT-2, MT-4, H9, PM1, CEM, myeloma cells (e.g., SB20 cells) and CEMX174 (such cell lines are available, for example, from the A.T.C.C.).

Similarly, bacterial hosts such as E. coli, Bacillus subtilis, and Streptococcus spp., will find use with the present expression constructs.

Yeast hosts useful in the present disclosure include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Fungal hosts include, for example, Aspergillus.

Insect cells for use with baculovirus expression vectors include, inter alia, Aedes aegypti, Autographa californica, Bombyx mori, Drosophila melanogaster, Spodoptera frugiperda, and Trichoplusia ni. See, Latham & Galarza (2001) J. Virol. 75(13):6154-6165; Galarza et al. (2005) Viral. Immunol. 18(1):244-51; and U.S. Patent Publications 200550186621 and 20060263804.

Cell lines expressing one or more of the sequences described above can readily be generated given the disclosure provided herein by stably integrating one or more expression vector constructs encoding the proteins of the VLP. The promoter regulating expression of the stably integrated influenza sequences (s) may be constitutive or inducible. Thus, a cell line can be generated in which one or more both of the matrix proteins are stably integrated such that, upon introduction of the sequences described herein (e.g., hybrid proteins) into a host cell and expression of the proteins encoded by the polynucleotides, non-replicating viral particles that present antigenic glycoproteins are formed.

In certain embodiments, a mammalian cell line that stably expressed two or more antigenically distinct RSV proteins is generated. Sequences encoding M1, M2 and/or additional glycoproteins (e.g., from the same or different virus strains) can be introduced into such a cell line to produce VLPs as described herein. Alternatively, a cell line that stably produces an M1 protein (and, optionally, M2) can be generated and sequences encoding the RSV protein(s) from the selected strain(s) introduced into the cell line, resulting in production of VLPs presenting the desired antigenic glycoproteins.

The parent cell line from which an VLP-producer cell line is derived can be selected from any cell described above, including for example, mammalian, insect, yeast, bacterial cell lines. In a preferred embodiment, the cell line is a mammalian cell line (e.g., 293, RD, COS-7, CHO, BHK, MDCK, MDBK, MRC-5, VERO, HT1080, and myeloma cells). Production of influenza VLPs using mammalian cells provides (i) VLP formation; (ii) correct post translation modifications (glycosylation, palmitylation) and budding; (iii) absence of non-mammalian cell contaminants and (iv) ease of purification.

In addition to creating cell lines, RSV-encoding sequences may also be transiently expressed in host cells. Suitable recombinant expression host cell systems include, but are not limited to, bacterial, mammalian, baculovirus/insect, vaccinia, Semliki Forest virus (SFV), Alphaviruses (such as, Sindbis, Venezuelan Equine Encephalitis (VEE)), mammalian, yeast and Xenopus expression systems, well known in the art. Particularly preferred expression systems are mammalian cell lines, vaccinia, Sindbis, insect and yeast systems.

Many suitable expression systems are commercially available, including, for example, the following: baculovirus expression (Reilly, P. R., et al., BACULOVIRUS EXPRESSION VECTORS: A LABORATORY MANUAL (1992); Beames, et al., Biotechniques 11:378 (1991); Pharmingen; Clontech, Palo Alto, Calif.)), vaccinia expression systems (Earl, P. L., et al., “Expression of proteins in mammalian cells using vaccinia” In Current Protocols in Molecular Biology (F. M. Ausubel, et al. Eds.), Greene Publishing Associates & Wiley Interscience, New York (1991); Moss, B., et al., U.S. Pat. No. 5,135,855, issued Aug. 4, 1992), expression in bacteria (Ausubel, F. M., et al., CURRENT PROTOCOLS N MOLECULAR BIOLOGY, John Wiley and Sons, Inc., Media Pa.; Clontech), expression in yeast (Rosenberg, S, and Tekamp-Olson, P., U.S. Pat. No. RE35,749, issued, Mar. 17, 1998, herein incorporated by reference; Shuster, J. R., U.S. Pat. No. 5,629,203, issued May 13, 1997, herein incorporated by reference; Gellissen, G., et al., Antonie Van Leeuwenhoek, 62(1-2):79-93 (1992); Romanos, M. A., et al., Yeast 8(6):423-488 (1992); Goeddel, D. V., Methods in Enzymology 185 (1990); Guthrie, C., and G. R. Fink, Methods in Enzymology 194 (1991)), expression in mammalian cells (Clontech; Gibco-BRL, Ground Island, N.Y.; e.g., Chinese hamster ovary (CHO) cell lines (Haynes, J., et al., Nuc. Acid. Res. 11:687-706 (1983); 1983, Lau, Y. F., et al., Mol. Cell. Biol. 4:1469-1475 (1984); Kaufman, R. J., “Selection and coamplification of heterologous genes in mammalian cells,” in Methods in Enzymology, vol. 185, pp 537-566. Academic Press, Inc., San Diego Calif. (1991)), and expression in plant cells (plant cloning vectors, Clontech Laboratories, Inc., Palo-Alto, Calif., and Pharmacia LKB Biotechnology, Inc., Pistcataway, N.J.; Hood, E., et al., J. Bacteriol. 168:1291-1301 (1986); Nagel, R., et al., FEMS Microbiol. Lett. 67:325 (1990); An, et al., “Binary Vectors”, and others in Plant Molecular Biology Manual A3:1-19 (1988); Miki, B. L. A., et al., pp. 249-265, and others in Plant DNA Infectious Agents (Hohn, T., et al., eds.) Springer-Verlag, Wien, Austria, (1987); Plant Molecular Biology: Essential Techniques, P. G. Jones and J. M. Sutton, New York, J. Wiley, 1997; Miglani, Gurbachan Dictionary of Plant Genetics and Molecular Biology, New York, Food Products Press, 1998; Henry, R. J., Practical Applications of Plant Molecular Biology, New York, Chapman & Hall, 1997).

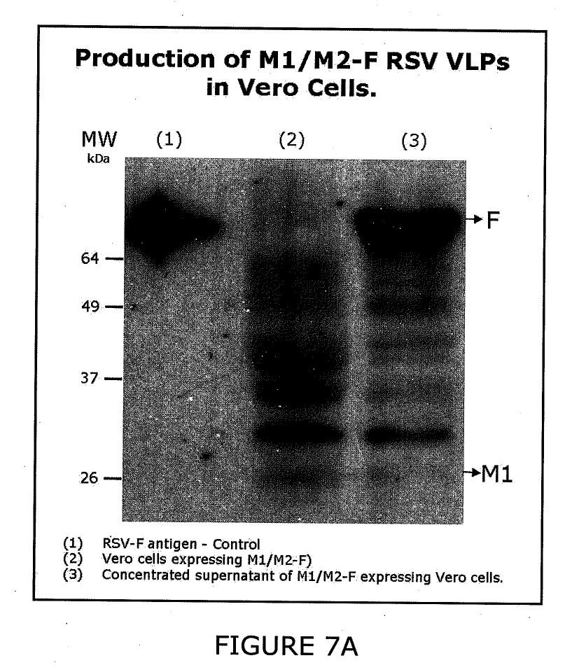

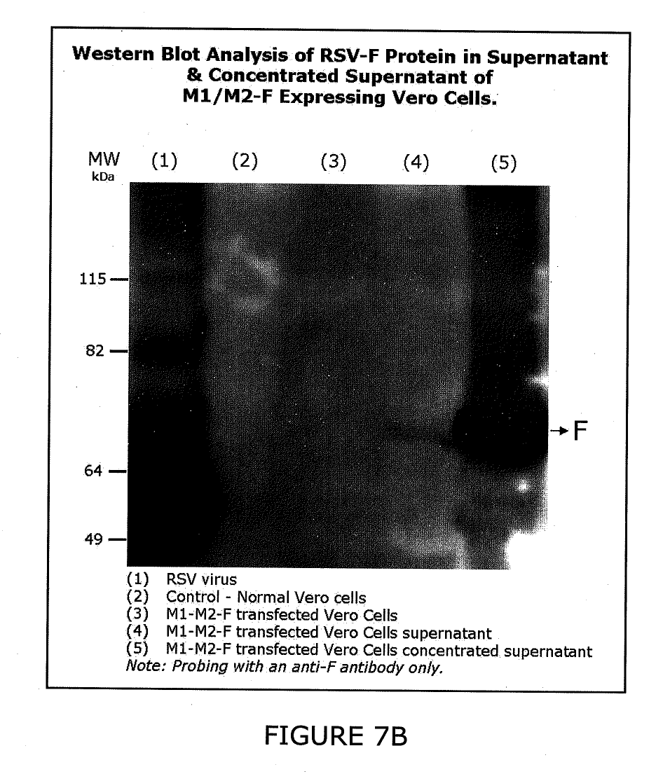

When expression vectors containing the altered genes that code for the proteins required for sub-viral structure vaccine formation are introduced into host cell(s) and subsequently expressed at the necessary level, the sub-viral structure vaccine assembles and is then released from the cell surface into the culture media (FIG. 7).

Depending on the expression system and host selected, the VLPs are produced by growing host cells transformed by an expression vector under conditions whereby the particle-forming polypeptide(s) is (are) expressed and VLPs can be formed. The selection of the appropriate growth conditions is within the skill of the art. If the VLPs are formed and retained intracellularly, the cells are then disrupted, using chemical, physical or mechanical means, which lyse the cells yet keep the VLPs substantially intact. Such methods are known to those of skill in the art and are described in, e.g., Protein Purification Applications: A Practical Approach, (E. L. V. Harris and S. Angal, Eds., 1990). Alternatively, VLPs may be secreted and harvested from the surrounding culture media.

The particles are then isolated (or substantially purified) using methods that preserve the integrity thereof, such as, by density gradient centrifugation, e.g., sucrose gradients, PEG-precipitation, pelleting, and the like (see, e.g., Kirnbauer et al. J. Virol. (1993) 67:6929-6936), as well as standard purification techniques including, e.g., ion exchange and gel filtration chromatography.

### Compositions

VLPs produced as described herein can be used to elicit an immune response when administered to a subject. As discussed above, the VLPs can comprise a variety of antigens (e.g., one or more RSV antigens from one or more strains or isolates). Purified VLPs can be administered to a vertebrate subject, usually in the form of vaccine compositions. Combination vaccines may also be used, where such vaccines contain, for example, other subunit proteins derived from influenza or other organisms and/or gene delivery vaccines encoding such antigens.

VLP immune-stimulating (or vaccine) compositions can include various excipients, adjuvants, carriers, auxiliary substances, modulating agents, and the like. The immune stimulating compositions will include an amount of the VLP/antigen sufficient to mount an immunological response. An appropriate effective amount can be determined by one of skill in the art. Such an amount will fall in a relatively broad range that can be determined through routine trials and will generally be an amount on the order of about 0.1 μg to about 10 (or more) mg, more preferably about 1 μg to about 300 μg, of VLP/antigen.

Sub-viral structure vaccines are purified from the cell culture media and formulated with the appropriate buffers and additives, such as a) preservatives or antibiotics; b) stabilizers, including proteins or organic compounds; c) adjuvants or immuno-modulators for enhancing potency and modulating immune responses (humoral and cellular) to the vaccine; or d) molecules that enhance Presentation of vaccine antigens to specifics cell of the immune system. This vaccine can be prepared in a freeze-dried (lyophilized) form in order to provide for appropriate storage and maximize the shelf-life of the preparation. This will allow for stock piling of vaccine for prolonged periods of time maintaining immunogenicity, potency and efficacy.

A carrier is optionally present in the compositions described herein. Typically, a carrier is a molecule that does not itself induce the production of antibodies harmful to the individual receiving the composition. Suitable carriers are typically large, slowly metabolized macromolecules such as proteins, polysaccharides, polylactic acids, polyglycollic acids, polymeric amino acids, amino acid copolymers, lipid aggregates (such as oil droplets or liposomes), and inactive virus particles. Examples of particulate carriers include those derived from polymethyl methacrylate polymers, as well as microparticles derived from poly(lactides) and poly(lactide-co-glycolides), known as PLG. See, e.g., Jeffery et al., Pharm. Res. (1993) 10:362-368; McGee J P, et al., J. Microencapsul. 14(2):197-210, 1997; O'Hagan D T, et al., Vaccine 11(2):149-54, 1993. Such carriers are well known to those of ordinary skill in the art.

Additionally, these carriers may function as immunostimulating agents (“adjuvants”). Exemplary adjuvants include, but are not limited to: (1) aluminum salts (alum), such as aluminum hydroxide, aluminum phosphate, aluminum sulfate, etc.; (2) oil-in-water emulsion formulations (with or without other specific immunostimulating agents such as muramyl peptides (see below) or bacterial cell wall components), such as for example (a) MF59 (International Publication No. WO 90/14837), containing 5% Squalene, 0.5% Tween 80, and 0.5% Span 85 (optionally containing various amounts of MTP-PE (see below), although not required) formulated into submicron particles using a microfluidizer such as Model 110Y microfluidizer (Microfluidics, Newton, Mass.), (b) SAF, containing 10% Squalane, 0.4% Tween80, 5% pluronic-blocked polymer L121, and thr-MDP (see below) either microfluidized into a submicron emulsion or vortexed to generate a larger particle size emulsion, and (c) Ribi™ adjuvant system (RAS), (Ribi Immunochem, Hamilton, Mont.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detoxu); (3) saponin adjuvants, such as Stimulon™. (Cambridge Bioscience, Worcester, Mass.) may be used or particle generated therefrom such as ISCOMs (immunostimulating complexes); (4) Complete Freunds Adjuvant (CFA) and Incomplete Freunds Adjuvant (IFA); (5) cytokines, such as interleukins (IL-1, IL-2, etc.), macrophage colony stimulating factor (M-CSF), tumor necrosis factor (TNF), beta chemokines (MIP, 1-alpha, 1-beta Rantes, etc.); (6) detoxified mutants of a bacterial ADP-ribosylating toxin such as a cholera toxin (CT), a pertussis toxin (PT), or an E. coli heat-labile toxin (LT), particularly LT-K63 (where lysine is substituted for the wild-type amino acid at position 63) LT-R72 (where arginine is substituted for the wild-type amino acid at position 72), CT-S109 (where serine is substituted for the wild-type amino acid at position 109), and PT-K9/G129 (where lysine is substituted for the wild-type amino acid at position 9 and glycine substituted at position 129) (see, e.g., International Publication Nos. WO93/13202 and WO92/19265); and (7) other substances that act as immunostimulating agents to enhance the effectiveness of the composition.

Muramyl peptides include, but are not limited to, N-acetyl-muramyl-L-threonyl-D-isoglutamine (thr-MDP), N-acteyl-normuramyl-L-alanyl-D-isogluatme (nor-MDP), N-acetylmuramyl-L-alanyl-D-isogluatminyl-L-alanine-2-(1′-2′-dipalmitoyl-sn-glycero-3-huydroxyphosphoryloxy)-ethylamine (MTP-PE), etc.

Examples of suitable immunomodulatory molecules for use herein include adjuvants described above and the following: IL-1 and IL-2 (Karupiah et al. (1990) J. Immunology 144:290-298, Weber et al. (1987) J. Exp. Med. 166:1716-1733, Gansbacher et al. (1990) J. Exp. Med. 172:1217-1224, and U.S. Pat. No. 4,738,927); IL-3 and IL-4 (Tepper et al. (1989) Cell 57:503-512, Golumbek et al. (1991) Science 254:713-716, and U.S. Pat. No. 5,017,691); IL-5 and IL-6 (Brakenhof et al. (1987) J. Immunol. 139:4116-4121, and International Publication No. WO 90/06370); IL-7 (U.S. Pat. No. 4,965,195); IL-8, IL-9, IL-10, IL-11, IL-12, and IL-13 (Cytokine Bulletin, Summer 1994); IL-14 and IL-15; alpha interferon (Finter et al. (1991) Drugs 42:749-765, U.S. Pat. Nos. 4,892,743 and 4,966,843, International Publication No. WO 85/02862, Nagata et al. (1980) Nature 284:316-320, Familletti et al. (1981) Methods in Enz. 78:387-394, Dull et al. (1989) Proc. Natl. Acad. Sci. USA 86:2046-2050, and Faktor et al. (1990) Oncogene 5:867-872); β-interferon (Seif et al. (1991) J. Virol. 65:664-671); γ-interferons (Watanabe et al. (1989) Proc. Natl. Acad. Sci. USA 86:9456-9460, Gansbacher et al. (1990) Cancer Research 50:7820-7825, Maio et al. (1989) Can. Immunol. Immunother. 30:34-42, and U.S. Pat. Nos. 4,762,791 and 4,727,138); G-CSF (U.S. Pat. Nos. 4,999,291 and 4,810,643); GM-CSF (International Publication No. WO 85/04188); tumor necrosis factors (TNFs) (Jayaraman et al. (1990) J. Immunology 144:942-951); CD3 (Krissanen et al. (1987) Immunogenetics 26:258-266); ICAM-1 (Altman et al. (1989) Nature 338:512-514, Simmons et al. (1988) Nature 331:624-627); ICAM-2, LFA-1, LFA-3 (Wallner et al. (1987) J. Exp. Med. 166:923-932); MHC class I molecules, MHC class II molecules, B7.1-β2-microglobulin (Parnes et al. (1981) Proc. Natl. Acad. Sci. USA 78:2253-2257); chaperones such as calnexin; and MHC-linked transporter proteins or analogs thereof (Powis et al. (1991) Nature 354:528-531). Immunomodulatory factors may also be agonists, antagonists, or ligands for these molecules. For example, soluble forms of receptors can often behave as antagonists for these types of factors, as can mutated forms of the factors themselves.

Nucleic acid molecules that encode the above-described substances, as well as other nucleic acid molecules that are advantageous for use within the present invention, may be readily obtained from a variety of sources, including, for example, depositories such as the American Type Culture Collection, or from commercial sources such as British Bio-Technology Limited (Cowley, Oxford England). Representative examples include BBG 12 (containing the GM-CSF gene coding for the mature protein of 127 amino acids), BBG 6 (which contains sequences encoding gamma interferon), A.T.C.C. Deposit No. 39656 (which contains sequences encoding TNF), A.T.C.C. Deposit No. 20663 (which contains sequences encoding alpha-interferon), A.T.C.C. Deposit Nos. 31902, 31902 and 39517 (which contain sequences encoding beta-interferon), A.T.C.C. Deposit No. 67024 (which contains a sequence which encodes Interleukin-1b), A.T.C.C. Deposit Nos. 39405, 39452, 39516, 39626 and 39673 (which contain sequences encoding Interleukin-2), A.T.C.C. Deposit Nos. 59399, 59398, and 67326 (which contain sequences encoding Interleukin-3), A.T.C.C. Deposit No. 57592 (which contains sequences encoding Interleukin-4), A.T.C.C. Deposit Nos. 59394 and 59395 (which contain sequences encoding Interleukin-5), and A.T.C.C. Deposit No. 67153 (which contains sequences encoding Interleukin-6).

Plasmids encoding one or more of the above-identified polypeptides can be digested with appropriate restriction enzymes, and DNA fragments containing the particular gene of interest can be inserted into a gene transfer vector (e.g., expression vector as described above) using standard molecular biology techniques. (See, e.g., Sambrook et al., supra, or Ausubel et al. (eds) Current Protocols in Molecular Biology, Greene Publishing and Wiley-Interscience).

### Administration

The VLPs and compositions comprising these VLPs can be administered to a subject by any mode of delivery, including, for example, by parenteral injection (e.g. subcutaneously, intraperitoneally, intravenously, intramuscularly, or to the interstitial space of a tissue), or by rectal, oral (e.g. tablet, spray), vaginal, topical, transdermal (e.g. see WO99/27961) or transcutaneous (e.g. see WO02/074244 and WO02/064162), intranasal (e.g. see WO03/028760), ocular, aural, pulmonary or other mucosal administration. Multiple doses can be administered by the same or different routes. In a preferred embodiment, the doses are intranasally administered.

The VLPs (and VLP-containing compositions) can be administered prior to, concurrent with, or subsequent to delivery of other vaccines. Also, the site of VLP administration may be the same or different as other vaccine compositions that are being administered.

Dosage treatment with the VLP composition may be a single dose schedule or a multiple dose schedule. A multiple dose schedule is one in which a primary course of vaccination may be with 1-10 separate doses, followed by other doses given at subsequent time intervals, chosen to maintain and/or reinforce the immune response, for example at 1-4 months for a second dose, and if needed, a subsequent dose(s) after several months. The dosage regimen will also, at least in part, be determined by the potency of the modality, the vaccine delivery employed, the need of the subject and be dependent on the judgment of the practitioner.

All patents, patent applications and publications mentioned herein are hereby incorporated by reference in their entireties.

Although disclosure has been provided in some detail by way of illustration and example for the purposes of clarity and understanding, it will be apparent to those of skill in the art that various changes and modifications can be practiced without departing from the spirit or scope of the disclosure. Accordingly, the foregoing disclosure and following examples should not be construed as limiting.

### EXAMPLES

### Example 1

### Generation of Mammalian Plasmids Carrying Single or Multiple Genes

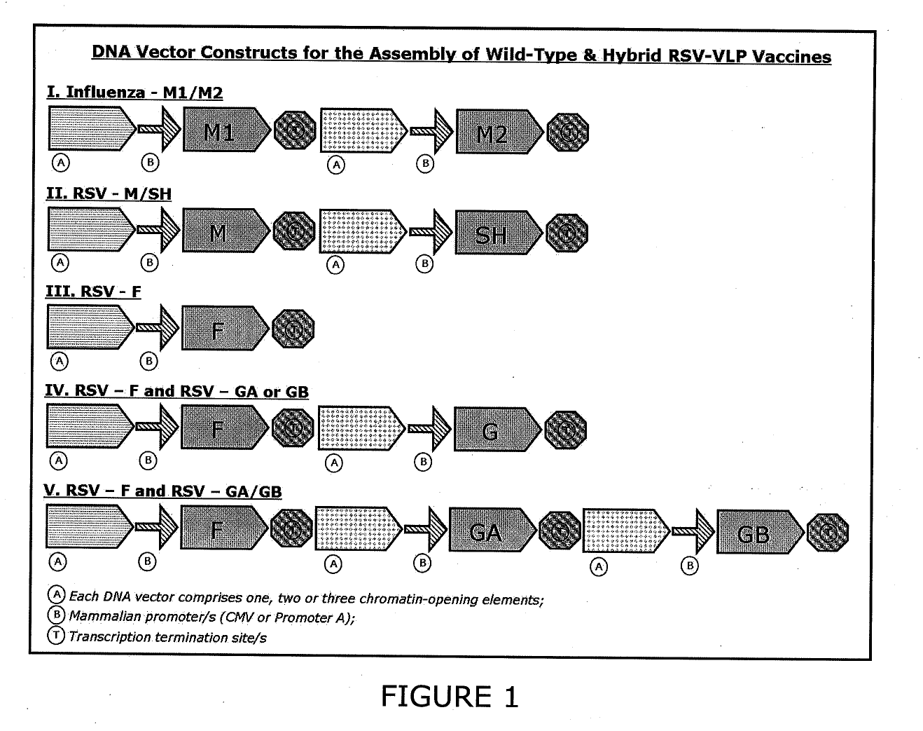

In this example, we describe the methods and materials needed to generate constructs required for the production of RSV-VLPs. The general structure of the constructs generated is shown in FIG. 1. The genes of interest were subcloned into mammalian plasmids (vectors) containing unique regulatory sequences that enhance not only high and sustainable levels of gene expression but also the rate of generating stably transfected cells.

The influenza M1 and M2 genes were sequentially subcloned into a mammalian plasmid expression vector using the appropriate restriction sites such that each gene was under the transcriptional control of a mammalian promoters (CMV promoter or promoter A). In addition, the plasmids into which the influenza M1 and M2 genes were cloned also contained an antibiotic selection markers (e.g., hygromicin, puromycin, or neomycin) and specific sequences upstream of each gene that maintain an open chromatin state following DNA integration within mammalian chromosomes.

The RSV genes were obtained by RT-PCR from RNA extracted from the respiratory syncytial virus A (RSV-A) Long strain and RSV-B Washigton strain (ATCC, Manassas, Va.). Genes were subcloned into an intermediate pET (NovaGen) or pGEM T (Promega) vector and subsequently amplified by PCR using specific primers that added unique restriction sites at each terminus. Using this strategy, the RSV-F was also subcloned into a mammalian vector as used for cloning of influenza matrix proteins to create an F only construct. A second RSV vector was created by adding the RSV-GA gene to F construct using the BstB1/NotI sites. A third RSV vector is generated by subcloning the RSV-B gene into the F-GA vector using the NaeI/BmtI. The RSV M/SH vector is generated by sequentially subcloning the RSV M and SH genes into the BstBI/NotI and NgoMIV/NheI respectively. After completion of each construct, the genes orientation and integrity were verified by restriction enzyme analysis and sequencing. Plasmid DNA of each construct was further amplified by transforming MAX Efficiency StbI2 competent E. Coli cells, and performing maxi-prep DNA preparation (Qiagen, Valencia, Calif.).

The concentration of plasmid DNAs were determined by spectrophotometry. Transient transfections were performed utilizing circular DNA, whereas transfections for the generation of stable cell lines were carried out with linear DNA cut with I-SceI restriction enzyme.

### Example 2

### Transfection of DNA Plasmids into Mammalian Cells by Electroporation

Following generation of the desired vectors as described in Example 1, the vectors were utilized for the production of VLPs in mammalian cells (CHO, Vero and MDCK).

To generate stably transfected cells linearized DNA was introduced to cell by electroporation. Briefly, 24 hours prior to DNA electroporation, selected cells were seeded into a T-175 flasks at a concentration of ˜4 million cells allowing for the formation of an 80-85% confluent monolayer in 24 hours. Prior to electroporation, the cell monolayer was washed once with 8 ml of 1×PBS (Gibco) then treated with 4 ml of 1× trypsin-EDTA (Gibco) and incubate for 5 minutes at 37° C. Cells were resuspended by adding to the flask 6 ml of DMEM (Gibco) containing 10% FBS (Invitrogen, San Diego, Calif.) collected in a tube and subsequently pelleted by centrifucation at 500×g for 5 minutes. Cell pellet was washed twice with 5 ml of ice cold 1×RPMI 1640 (Cellgro, Mediatech, Manassas, Va.) and then resuspend in 5000 of ice cold 1×RPMI.

The cell suspension received 6 μg of linearized plasmid expressing M1/M2, gently mixed by pipeting and then transferred into a 0.4 cm gap electroporation cuvette (Bio-Rad, Hercules, Calif.). The cuvette was placed in a Bio-Rad Gene Pulsar, and cells electroporated using the following parameters: 400V, 960 μF. Electroporated cells were kept at room temperature for 5 minutes, then transferred into a 6-well plate in DMEM with 10% FBS and penicillin/streptomycin (Gibco) and incubated at 37° C. with 5% CO2. Six hours post electroporation, the medium was aspirated, cells washed once with 1×PBS, and fresh medium added. Cells were incubated at 37° C. with 5% CO₂ until antibiotic selection was initiated.

A modified protocol was used for the electroporation of suspension cells, e.g. CHO cell line. Cells were directly collected from the culture vessel without the need of trypsin treatment. Subsequent steps were performed as described above.

### Example 3

### Transfection of DNA Plasmids into Mammalian Cells by Chemical Methods

Mammalian cells (CHO, Vero, or MDCK) were prepared for transfection by plating in an appropriate culture vessel (25 cm2 flasks for CHO cells or 75 cm2 flasks for Vero and MDCK cells) at a density of 1.5×10⁶ to 2.5×10⁶ cells/ml in 5 ml of CHO—S—SFM II medium (CHO cells) or 10 ml of DMEM (Vero, MDCK) supplemented with 5% fetal bovine serum (FBS) (Invitrogen, Carlsbad, Calif.). Aherent cells (Vero, MDCK) were plated 24 hours prior to the initiation of the transfection procedure.

Following this step, a DNA-lipid complexing reaction comprising of plasmid DNA with lipofectamine was set up. The plasmid DNA of interest or mixture thereof was diluted in 500 μl of Opti-MEM medium in one tube and 20 μl of lipofectamine 2000 was diluted in 480 μl of Opti-MEM medium (Invitrogen, Carlsbad, Calif.) in another tube. The lipofectamine-OptiMEM mixture was incubated at room temperature for 5 minutes.

After this step, the plasmid DNA-OptiMEM mixture was combined with the lipofectamine-OptiMEM mixture and the reaction was allowed to proceed at room temperature for 20 minutes. The DNA-lipofectamine complex was then added to the cells previously plated as described above. In the case of CHO cells, five hours after the addition of the DNA-lipofectamine complex, 2.5 ml of the contents of the 25 cm2 flask was transferred to another 25 cm2 flask and 4.5 ml of CHO—S-SFM II medium was added to both flasks. Adherent cells were kept in the culture flask and 5 ml of fresh media was added. None of the reagents or media used in the transfection process contained any antibiotics as these could get delivered to the interior of the cells by getting incorporated in the DNA-lipid complex which could prove toxic to the cells.

For the transient expression of the proteins of interest to create VLPs, the plasmids were introduced into the cells in the form of a DNA-lipid complex in which the DNA is in its native circular form. Expression of the VLP proteins in the transfected cell lysate and in the culture supernatant was evaluated 72-96 hours post-transfection.

To generate stably transfected cells, in which the plasmid/s of interest are integrated with the chromosomal cellular DNA establishing an state of continuous protein expression, the transfection procedure was followed with the exception that the plasmid/s introduced into the cells were in a linear configuration. The plasmids into which the genes of interest were cloned also contain antibiotic resistance markers e.g. Hygromycin, Puromycin or Neomycin. Thus, after 48-96 hours post transfection, cells were subjected to selection by adding to the culture medium the antibiotic of choice based on the resistance gene delivered by the plasmid. Following 8-10 days of selection, cells that grew in the presence of the antibiotic were isolated based on the expression and detection of surface protein/s using fluorescence-activated cell sorting (FACS).

### Example 4

### Transfection of Circular DNA for Transient Expression of Protein and RSV-VLP Assembly

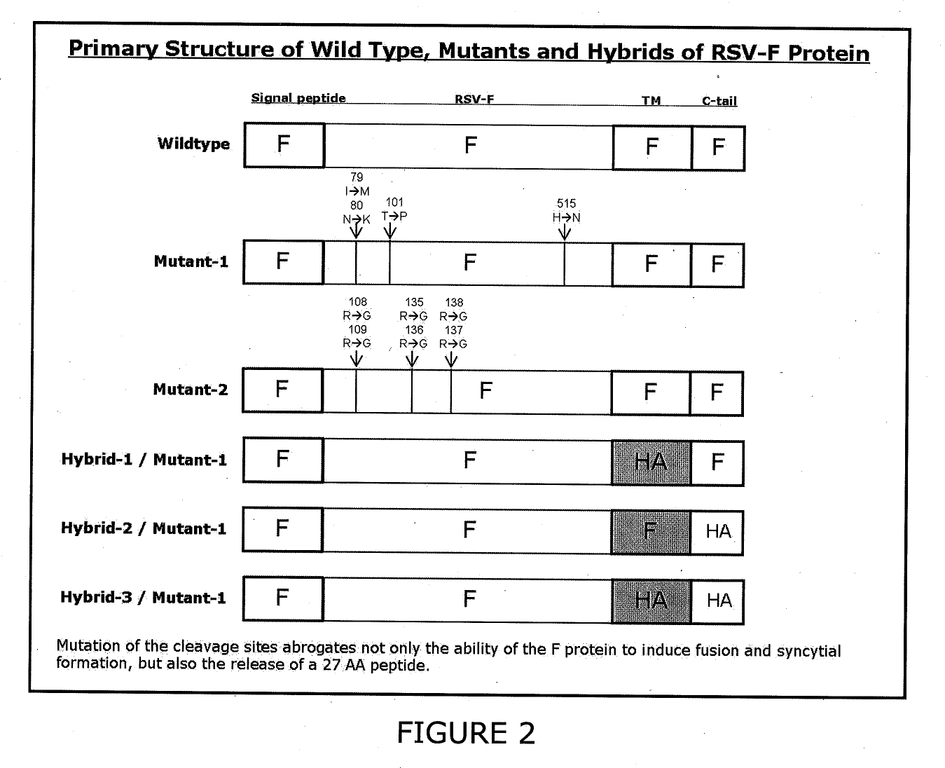

Assessment of RSV VLPs production was performed by introducing a combination of uncut circular plasmid to mammalian cells by either electroporation or chemical transfections. In this example the M1/M2 vector was combined with RSV-F (mutant 1, FIG. 2) and delivered to Vero cells by either of the two transfection methods described.

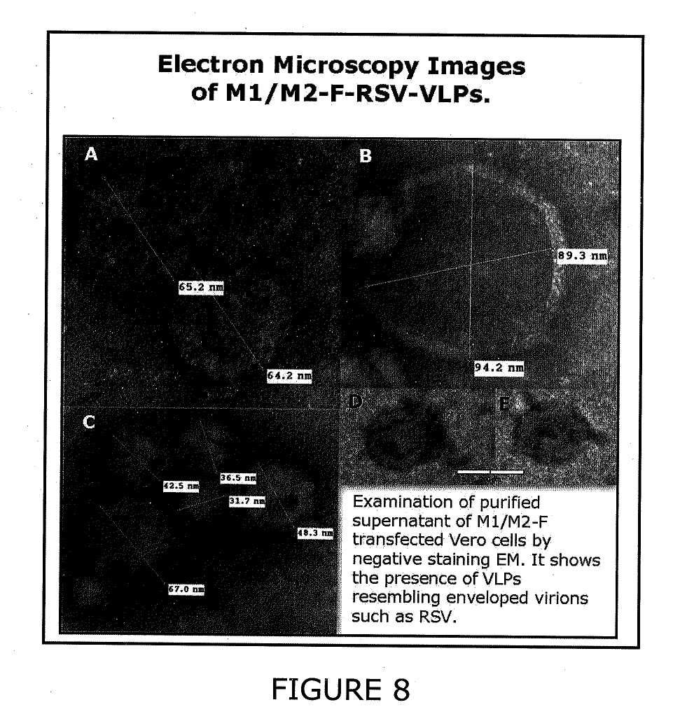

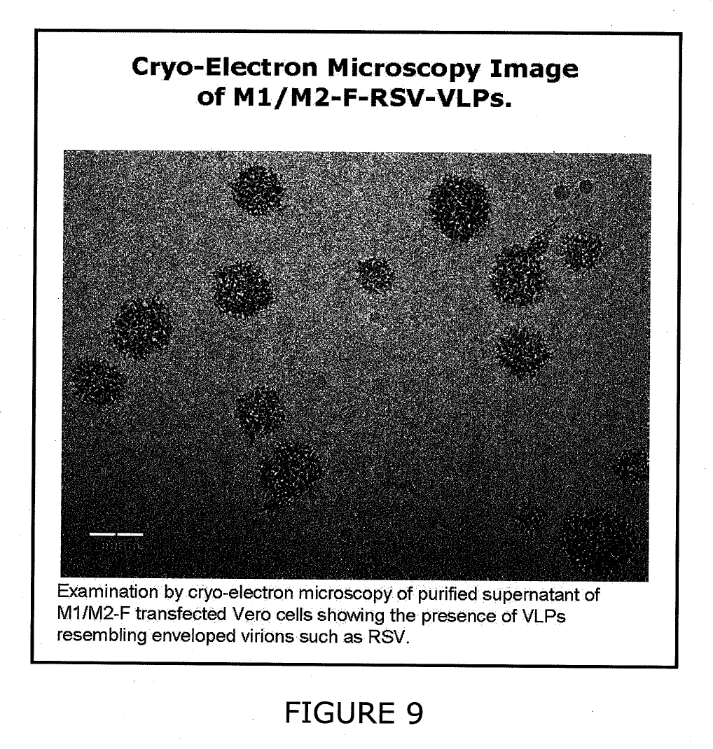

Western blot analysis of cell lysates, culture supernatant and concentrated purified supernatant demonstrated that these proteins were expressed in the cells and released into the culture media as shown in FIGS. 7A and 7B. Furthermore, concentrated and purified material produced and released for these cells was examined by negative staining electron microscopy and cryo-electron microscopy. These studies revealed the presence of virus-like particles which resemble enveloped virus particles such as the RSV virus (FIG. 8 and FIG. 9)

### Example 5

### Transfection of Linearzied DNA to Generate Stably Transfected Mammalian Cells

Transfection of linearized plasmid DNA into mammalian cells leads to plasmid integration into the host cell genome. Delivery of linear forms of any of the constructs described on FIG. 1 leads to plasmid integration and subsequent expression the genes carried by the vector DNA. This strategy allows for the creation of stably transfected cell lines that constitutively express the gene product/s of interest.

Pursuing this approach, a linearized M1/M2 vector (FIG. 1-I) was introduced by electroporation or chemical transfection into MDCK, Vero or CHO cells. After this step, cells were treated with one antibiotic (hygromicin, puromycin or neomycin) chosen based on the antibiotic resistance gene carried by the plasmid. The M1/M2 vector contains the hygromycin resistance gene, thus cells transfected with this plasmid were treated with this antibiotic to select stably transfected cell lines. Cells that grew in the presence of the antibiotic were tested for the expression of the M2 protein on their surface and cloned using fluorescence activated cell sorting (FACS).

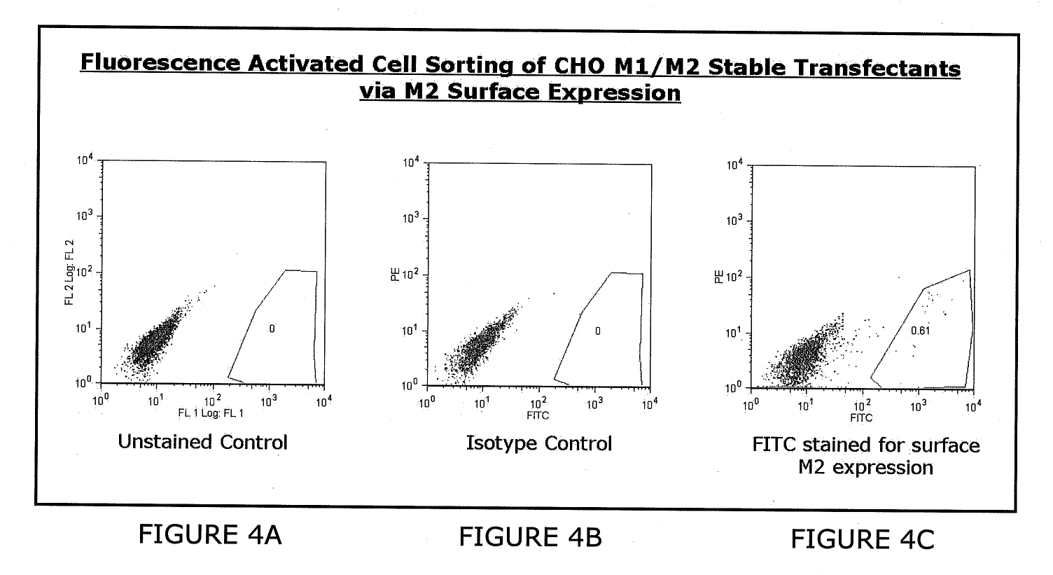

FIG. 4 shows FACS histogram of M1/M2 transfected CHO cells which demonstrated that fraction of cell population expressed the M2 protein. Multiple cell clones were expanded and the expression of both M1 and M2 proteins further evaluated by Western blot.

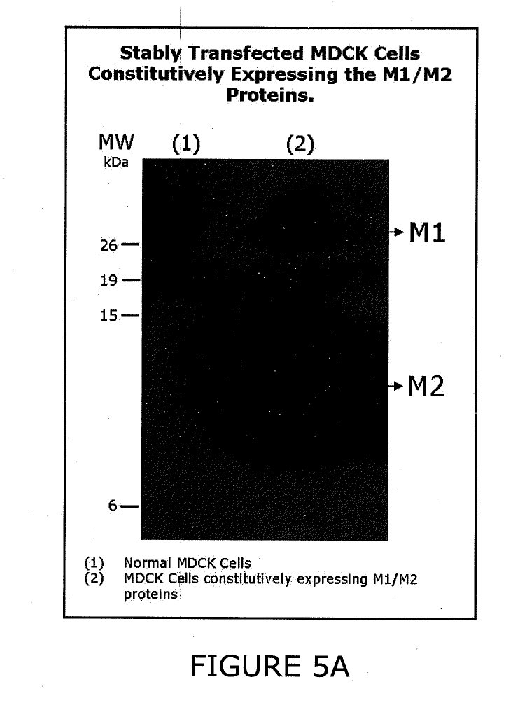

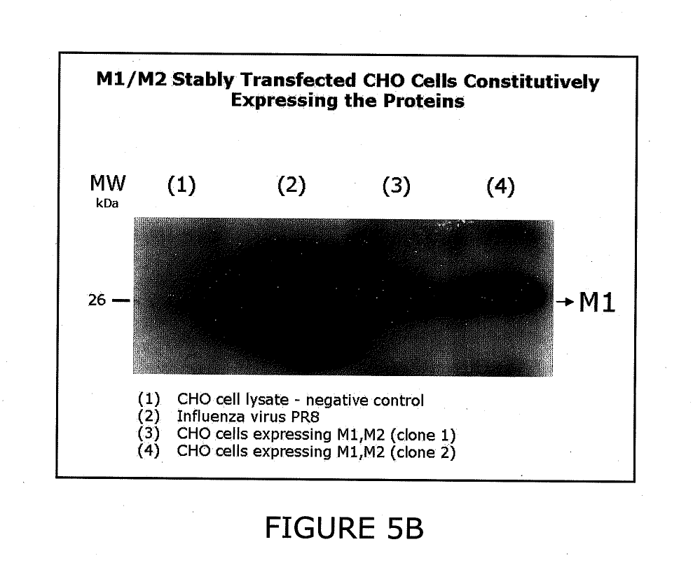

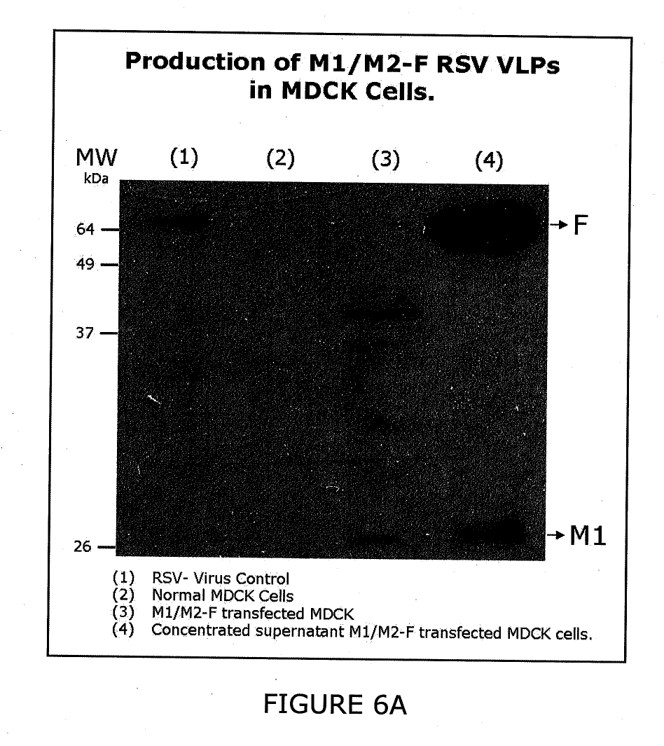

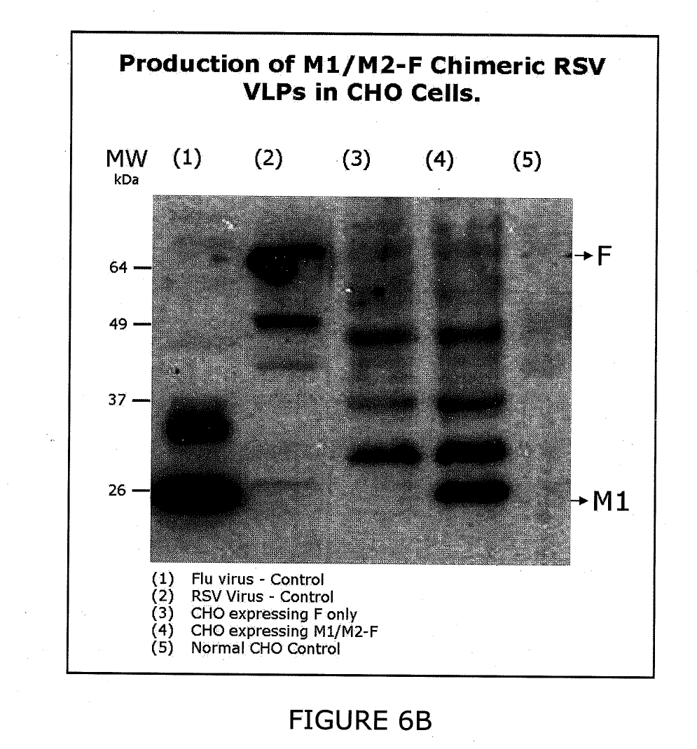

Furthermore, as shown in FIGS. 5A and 5B, selected M1/M2 stably transfected MDCK and CHO cell lines constitutively express these proteins. These cell lines were transfected with the RSV-F vector for the assembly and release of RSV virus-like particles as shown FIGS. 6A, and 6B.

### Example 6

### Selection of Stably Transfected Mammalian Cells

Mammalian cells that had been transfected with linearized plasmids encoding gene(s) of interested were then subjected to antibiotic treatment to select out the cells in which the transfected plasmid had integrated into the host cell genome. The transfected plasmid contains an antibiotic resistance cassette which confers resistance to either hygromycin, puromycin or neomycin.

About 48-72 hours post-transfection cells were subjected to selection pressure by adding the appropriate concentration of antibiotic (400 ug/ml of hygromycin, 10 ug/ml of puromycin or 10 ug/ml of neomycin) to the cell culture medium and sub-culturing the transfected cells in this medium through several cell passages. This process allowed the survival of only those cells into which chromosomal integration of the genes from the transfected plasmid has taken place and were selected out from the rest of the population.

### Example 7

### Fluorescence Activated Cell Sorting (FACS) Analysis of M1/M2 Transfected Cells

Cells obtained as described above were harvested from the tissue culture flasks and washed three times by consecutive centrifugation at 500×g for 5 min followed by re-suspension in phosphate buffered saline (PBS). After the third wash, cells were counted and divided into three aliquots of 5×10⁶ cells each. One aliquot, the one which was going to be sorted for cell surface M2 expression, was incubated with an anti-M2 monoclonal antibody (Abeam Inc, Cambridge, Mass.) at a dilution of 1:500 in PBS for 1 hour at room temperature. The second aliquot was incubated with a monoclonal antibody to influenza A nucleoprotein (Meridian Life Sciences, Saco, Me.) at a dilution of 1:200 in PBS for 1 hour at room temperature. This aliquot served as an isotype control for the flow cytometry experiment. The third aliquot served as the unstained control for the experiment.

After the incubation with the respective antibodies, the cells were washed with PBS three times and incubated with a Fluorescein isothiocyanate (FITC) labeled anti-mouse antibody (Abeam Inc, Cambridge, Mass.) at a dilution of 1:100 in PBS for 1 hr at room temperature. After these treatments, the cells were washed three times with PBS and re-suspended in a final volume of 3 ml of cell culture medium. These samples were then analyzed in a MoFlo cell sorter.

Cells that expressed high levels of M2 protein on their cell surface, as determined by their specific green fluorescence spectrum were sorted as 1 cell/well into 96 well plates that contained 100 μl of cell culture medium in each well. FIG. 4 depicts a sorting experiment performed with M1/M2 transfected cells.

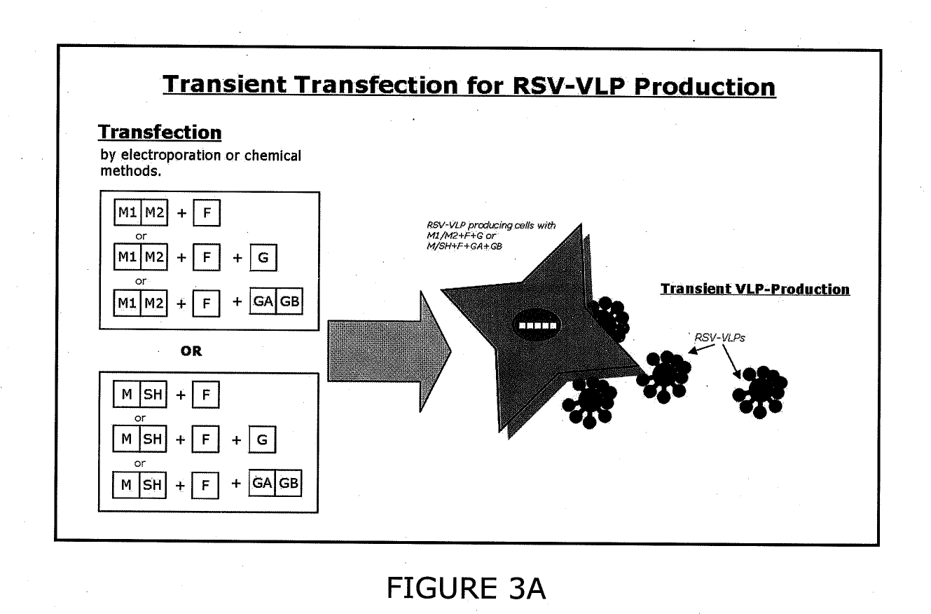

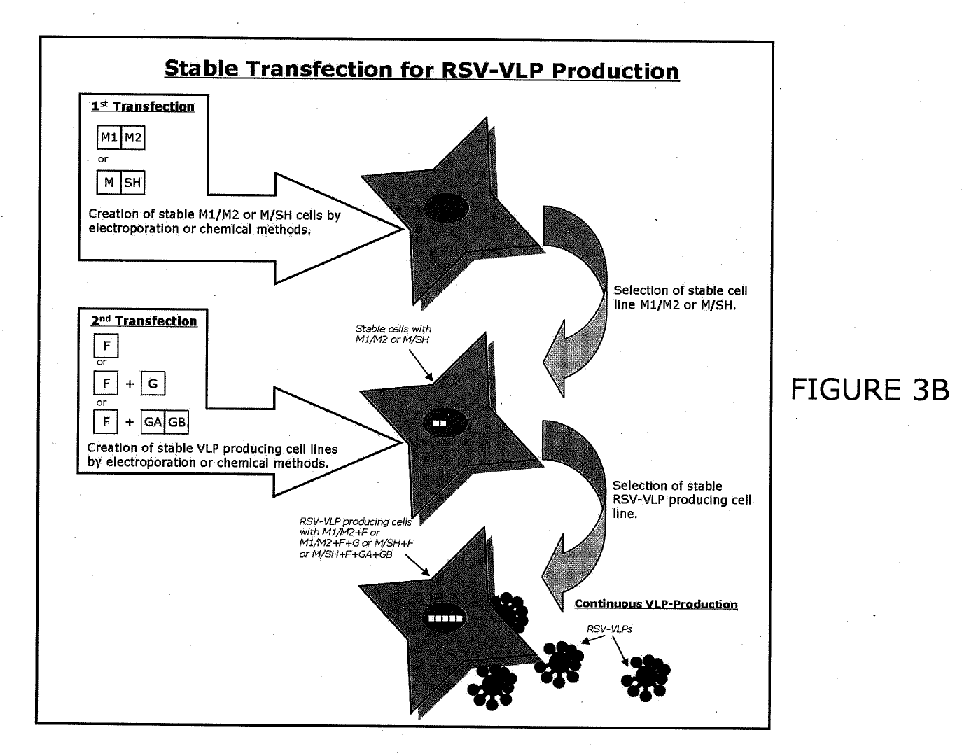

This selection method is applied for the identification and isolation of cells transfected with any the constructs described on FIG. 1. Cells transfected with the DNA vector carrying the RSV M and SH, FIG. 1 (FIG. 1.II) express both M and SH protein, one of which the SH is displayed on the cell surface allowing for the identification and isolation of stably transfected clones that continuously express these proteins. Following the creation of the first stably transfected cell line, a second transfection is performed to introduce and integrate another set of genes into the host genome of the basic cell line. This strategy is depicted in FIG. 3B, and identification and selection of stably cell lines generated on the second round of transfections is also performed by FACS analysis. The three RSV proteins F, GA and GB which are delivered by any of the three vector constructs depicted on FIG. 1 (constructs shown in panels III-IV-or V) are surface molecules and displayed on the cell membrane, therefore suitable for the FACS strategy to identify and selected stably transfected cell lines that continuously produce RSV VLPs.

### Example 8

### End-Point Cloning and Expansion of a Stably Transfected and Constitutively Producing M1 and M2 Influenza Proteins

To obtain clonal cell lines from the stably transfected cell population, end point cloning was performed. The cells were harvested and counted using a hemocytometer. The cells were then diluted in an appropriate volume of culture medium such that the final concentration reached 10 cells per ml. This cell preparation was gently agitated to ensure homogenous cell distribution and then plated into sterile 96 well plates at 100 μl/well. The plates were then incubated at 37° C. with a humidified atmosphere of 5% CO2 and monitored regularly for clonal cell growth. Wells with actively growing cells were identified over a period of time. When the clonal cells in these wells reached about 70-80% confluency they were scaled up by sequential passages to 6 well plates, 25 cm² and 75 cm² flasks.

### Example 9

### Analysis of Protein Expression

Analysis of protein expression was carried out on cell pellets and on supernatants of transfected mammalian cells. Tranfected cells were harvested, washed with 1×PBS and resuspended in RIPA buffer [Tris-HCl (pH 7.4) 50 mM, Sodium Chloride 150 mM, EDTA 1 mM, Triton X-100 1%, NP-40 1%, Sodium dodecyl sulfate 0.1%] and then disrupted by freezing and thawing cycles and pipeting and finally stored at −20° C. until further used. The total protein concentration in each sample was estimated by the Bradford method; briefly, 10 μl of the sample is added to 1.0 ml of 1× Bradford Dye reagent (Bio-Rad Inc., Hercules, Calif.) which was pre-warmed to room temperature. This reaction mixture is then shaken vigorously to create a homogenous solution. The absorbance of each sample was measured in a spectrophotometer at a wavelength of 595 nm. The protein concentration of each sample was determined using an standard curve which was plotted by measuring absorbance at 595 nm of known concentrations of bovine serum albumin using the Bradford assay.

For western blot analysis equivalent amount of protein were loaded onto an SDS-PAGE and resolved by electrophoresis at 125V for 1.5 hours. Subsequently, protein were electroblotted onto a nitrocellulose or PVDF membranes and subjected to western blot procedures consisting of the following steps: blocking, incubation with primary antibody/ies, washings, incubation with horseradish peroxidase conjugated-secondary antibody, washing again and finally reaction with chemioluminescence substrate and signal detection.

### Example 10

### Selection of a Cell Line that Continuously Expresses the Influenza M1 and M2 Proteins

MDCK and CHO cells were transfected by electroporation with a linearized DNA vector carrying the M1 and M2 influenza genes. Following selection with hygromycin, single cell clones were isolated using fluorescence-activated cell sorting (FACS) after labeling the cells with combination of antibodies; first as primary an anti-M2 mouse monoclonal antibody which reacted the M2 protein expressed on the cell surface, followed by a flourescein conjugated anti-mouse as secondary.

Sorted single cells were expanded and expression of M1 and M2 proteins was further assessed by Western blot. Cells were lysed, loaded onto and SDS-PAGE (10-20%) and separated by electrophoresis. The proteins were transferred to a PVDF membrane and blocked with 4% skim milk for 1 hour. The membrane was then incubated overnight with a mouse monoclonal anti-M1 protein (1:40,000 dilution) and a mouse monoclonal anti-M2 protein (1:1000 dilution) (Abeam, clone 14C2). The membrane was washed 3× for 5 minutes with 1× TBST and then incubated for 1 hour with horseradish peroxidase conjugated goat anti-mouse IgG (1:50,000 dilution in 2% skim milk) (Thermo Scientific, Rockford, Ill.). The membrane was washed 3× for 10 minutes with 1× TBST followed by 5 minute incubation with SuperSignal West Pico chemiluminescent substrate (Thermo Scientific, Rockford, Ill.). The membrane was exposed to HyBlot CL autoradiography film (Denville Scientific, Metuchen, N.J.).

As shown in FIG. 5A (lane 3), the MDCK cell lysate produced two major bands which correspond to the M1 protein molecular weight (MW) of ˜27 kDA and M2 protein with a MW of ˜11 kDA, whereas untransfected control did not show the presence of these proteins. Lane 1: protein marker, lane 2: MDCK cells not expressing influenza M1 and M2 proteins (negative control), lane 3: MDCK cells constitutively expressing influenza A M1 and M2 proteins. Similar results were obtained with the same plasmid was introduced into CHO cells as shown in FIG. 5B and VERO cells.

### Example 11

### Expression of RSV-F Protein in M1/M2 Producing Cells

Transfection of a DNA vector carrying the wild type or mutated RSV-F (FIG. 1-Construct III) together with the M1/M2 DNA vector (FIG. 1-Construct I) or into cells already expressing the M1/M2 led to the expression of these proteins which were not only present in cell lysates but also in the cell supernatant and concentrated purified supernatant.

Western blot analysis of these samples are shown in FIG. 6 and show production of M1, M2 and RSV F protein in the transfected cells.

### Example 12

### Examination of RSV-VLP by Negative Staining Electron Microscopy

The supernatant of the transfected cells was subjected to ultra-centrifugation at 200,000×g for 1.5 hours at 4° C. The pellet from the ultra-centrifugation was re-suspended in PBS and 20 μl of the re-suspended pellet was set aside for electron microscopy. This 20 μl sample was fixed with 4% para-formaldehyde and then 5 μl of the fixed material was applied to a 200 mesh carbon coated grid (EMS, Hatfield, Pa.) and allowed to cover the grid for 5 minutes and then washed with water three times. Following this the coated grids were exposed to five drops of 2% uranyl acetate in quick succession to prevent over-staining of the grid with uranyl acetate. Excessive solution was blotted from the grid and then air dried before loading it onto a JOEL JEM 100CX Transmission Electron Microscope. The samples were observed at a magnification of 60,000× to 100,000×.

FIG. 8 shows images obtained from purified supernatant of M1/M2-F transfected Vero cells. FIG. 9 shows micrograph images of unfixed material processed for cryo-electron microscopy examination.

### Example 13

### In vivo vaccination with RSV VLPs

The immunogenicity and protective efficacy of RSV VLP vaccines are tested in BALB/C mice and cotton rats (Charles River Laboratories, Wilmington, Mass.). Mice or cotton rats are immunized intranasally and/or or intramuscularly with single or multiple doses of VLPs as described herein.

What is claimed is:

1. A virus-like particle (VLP) comprising
an M protein, wherein the M protein is an influenza M1 protein or an RSV M protein;
an M2 protein, wherein the M2 protein is an influenza M2 protein or an RSV M2 protein; and
a respiratory syncytial (RSV) F protein,
and further wherein if the VLP does not contain an influenza protein, the VLP further comprises an RSV SH protein.

2. The VLP of claim 1, further comprising one or more additional RSV proteins.

3. The VLP of claim 2, comprising at least one RSV G protein.

4. The VLP of claim 3, wherein the RSV G protein is selected from the group consisting of (i) one more Group A RSV G proteins; (ii) one or more Group B RSV G proteins; and (iii) one or more Group A and one more Group B RSV G proteins.

5. The VLP of claim 3, wherein the RSV G protein is selected from the group consisting of a wild-type RSV G protein, an RSV G protein comprising a mutation in the central domain and combinations thereof.

6. The VLP of claim 1, wherein the RSV F protein is selected from the group consisting of a wild-type RSV F protein, an RSV F protein comprising one more amino acid modifications as compared to wild-type, a hybrid RSV F protein and combinations thereof.

7. The VLP claim 6, wherein the one or more amino acid modifications to the RSV F protein are selected from the group consisting of additions, deletions, replacements and combinations thereof.

8. The VLP of claim 7, wherein the modifications inhibit cleavage of the F protein into F1 and F2.

9. The VLP of claim 8, wherein the modifications inhibit membrane fusion.

10. The VLP of claim 6, wherein the RSV F protein is a hybrid protein, the hybrid protein comprising RSV F sequences and influenza sequences.

11. The VLP of claim 10, wherein the hybrid protein comprises an RSV F protein and a transmembrane domain of an influenza HA protein.

12. The VLP of claim 10, wherein the hybrid protein comprises an RSV F protein and a cytoplasmic tail domain of an influenza HA protein.

13. The VLP of claim 12, wherein the hybrid protein comprises an RSV F protein and a transmembrane domain of an influenza HA protein.

14. The VLP of claim 1, wherein the VLP is expressed in a eukaryotic cell under conditions which permit the assembly and release of VLPs.

15. The VLP of claim 14, wherein the eukaryotic cell is selected from the group consisting of a yeast cell, an insect cell, an amphibian cell, an avian cell, a plant cell or a mammalian cell.

16. The VLP of claim 2, wherein the additional RSV proteins are selected from the group consisting of one or more RSV M proteins, one or more RSV SH proteins, and combinations thereof.

17. A method of producing a VLP, the method comprising the steps of
transfecting one or more vectors encoding two M proteins and an RSV F protein into a suitable host cell and expressing the combination of protein under conditions that allow VLP formation.

18. The method of claim 17, wherein at least one M protein comprises an influenza matrix protein.

19. The method of claim 17, wherein at least one M protein comprises an RSV M protein.

20. The method of claim 17, wherein the one or more vectors are stably transfected into the host cell.

21. The method of claim 17, wherein the M proteins and F proteins are encoded on separate vectors and further wherein the vector encoding the M proteins is stably transfected into the cell prior to transfection with the vector encoding the RSV F protein.

22. The method of claim 17, wherein the one or more vectors further encode an RSV G protein.

23. The method of claim 17, wherein the one or more vectors further encode an RSV SH protein.

24. The method of claim 17, wherein the RSV F protein is selected from the group consisting of a wild-type RSV F protein, an RSV F protein comprising one more amino acid modifications or truncations as compared to wild-type, a hybrid RSV F protein and combinations thereof.

25. The method of claim 22, wherein the RSV G protein is selected from the group consisting of a wild-type RSV G protein, an RSV G protein comprising a mutation in the central domain and combinations thereof.

26. The method of claim 17, wherein the cell is a eukaryotic cell selected from the group consisting of a yeast cell, an insect cell, an amphibian cell, an avian cell, a plant cell or a mammalian cell.

27. An immunogenic composition comprising at least one VLP according to claim 1.

28. The immunogenic composition of claim 27, further comprising an adjuvant.

29. The immunogenic composition of claim 27, wherein the composition comprises at least two VLPs comprising different RSV proteins.

30. A method of generating an immune response to RSV in a subject, the method comprising administering to the subject an effective amount of the immunogenic composition according to claim 27.

31. The method of claim 30, wherein the composition is administered mucosally, intradermally, subcutaneously, intramuscularly, or orally.

32. The method of claim 30, wherein the immune response vaccinates the subject against RSV.

33. The method of claim 30, wherein the subject is a human.
